SEQUENCE LISTING (4 sequences)

SEQ ID NO: 1 - DNA, 87 bp, Artificial Sequence
  Description of Artificial Sequence Synthetic oligonucleotide
gcctgttgtgagcctcctgtcgaannnnnnnnnnnnnnnnnnnnnnnnnnnnnnnnnnnnnnnnttgagcgtttattcttgtctccc

SEQ ID NO: 2 - DNA, 24 bp, Artificial Sequence
  Description of Artificial Sequence Synthetic primer
gcctgttgtgagcctcctgtcgaa

SEQ ID NO: 3 - DNA, 23 bp, Artificial Sequence
  Description of Artificial Sequence Synthetic primer
gggagacaagaataaacgctcaa

SEQ ID NO: 4 - DNA, 92 bp, Artificial Sequence
  Description of Artificial Sequence Synthetic oligonucleotide
gcctgttgtgagcctcctgtcgaannnnnnnnnnnnnnnnnnnnnnnnnnnnnnnnnnnnnnnnnnnnnttgagcgtttattcttgtctccc

(19) United States
(12) Patent Application Publication
(10) Pub. No.: US 2017/0067091 A1
(43) Pub. Date: Mar. 9, 2017

(54) METHODS AND DEVICES FOR SELECTION AND ISOLATION OF APTAMERS

(71) Applicant: THE TRUSTEES OF COLUMBIA UNIVERSITY IN THE CITY OF NEW YORK, New York, NY (US)

(72) Inventors: Qiao Lin, New York, NY (US); Jing Zhu, Beijing (CN); Timothy Olsen, New York, NY (US)

(21) Appl. No.: 15/269,494

(22) Filed: Sep. 19, 2016

Related U.S. Application Data
(63) Continuation of application No. PCT/US2015/022044, filed on Mar. 23, 2015.
(60) Provisional application No. 61/968,919, filed on Mar. 21, 2014.
(60) Provisional application No. 62/068,554, filed on Oct. 24, 2014.
(60) Provisional application No. 62/109,596, filed on Jan. 29, 2015.

(51) Int. Cl. C12Q 1/68; B01L 7/00; B01L 3/00

(52) U.S. Cl. CPC C12Q 1/6811; B01L 3/5027; B01L 7/52; B01L 2200/0668; B01L 2200/10; B01L 2300/0816; B01L 2300/088; B01L 2300/1827; B01L 2400/0421; B01L 2400/0481; B01L 2400/0655

(57) ABSTRACT

A microdevice for isolating and amplifying aptamers includes a selection microchamber and an amplification microchamber. The selection microchamber can include a plurality of cultured cells immobilized therein. A first microchannel connecting the selection microchamber to the amplification microchamber can be configured to hydrodynamically transfer oligomers from the selection microchamber to the amplification chamber. A second microchannel connecting the selection microchamber to the amplification microchamber can be configured to hydrodynamically transfer oligomers from the amplification chamber to the selection chamber.

### CROSS REFERENCES TO RELATED APPLICATIONS

This application is a continuation of International Patent Application No. PCT/US2015/022044, filed Mar. 23, 2015, which claims priority from U.S. Provisional Application No. 61/968,919, filed Mar. 21, 2014; Provisional Application No. 62/068,554, filed Oct. 24, 2014; and Provisional Application No. 62/109,596, filed Jan. 29, 2015, each of which is incorporated by reference herein in its entirety.

### SEQUENCE LISTING

The instant application contains a Sequence Listing which has been submitted in ASCII format via EFS-Web and is hereby incorporated by reference in its entirety. Said ASCII copy, created on Nov. 17, 2016, is named 070050_5798_SL.txt and is 1,696 bytes in size.

### STATEMENT REGARDING FEDERALLY FUNDED RESEARCH

This invention was made with government support under CBET-0854030, awarded by the National Science Foundation; 8R21GM104204, 5U19 AI067773, RR025816-02, and CA 14 7925-01 awarded by the National Institute of Health. The government has certain rights in this invention.

### BACKGROUND

Chemical amplification of nucleic acids can be realized with polymerase chain reaction (PCR), in which a DNA molecule (a template) can be duplicated via repeated thermal denaturation and enzymatic replication. Bead-based PCR is a variant of PCR that uses primers (short DNA fragments complementary to a specific region of the template) attached to microbeads. This procedure can result in bead-tethered template DNA duplicates, and can serve as an analytical tool to simultaneously accumulate signals from DNA-based transducers and allow manipulation of DNA itself via solid-phase extraction (SPE) techniques.

Bead-based PCR has been used in DNA sequencing, protein screening, and pathogenic DNA detection. For example, certain whole genome sequencing has been performed using bead-based PCR to facilitate the organization and detection of amplified sections of a fragmented E. coli genome. Compartmentalization of DNA in emulsions combined with bead-based PCR can allow for rapid screening of an entire genome for DNA binding proteins and cell-free protein synthesis.

Microfluidics technology can provide a rapid and efficient reaction platform due to efficient heat transfer properties. Microfluidics can also be used to provide integrated chip-based systems that perform tasks such as sample pretreatment and post-amplification analysis.

In bioanalytical assays, analytes of interest can be present in minute quantities and contaminated with impurities. Sample preparation prior to analysis can be important for improving the resolution of detection results. For example, isolation and enrichment of DNA molecules within dilute and complex samples can be used for clinical detection of DNA markers linked to disease and synthetic selection of analyte-specific molecules such as aptamers.

Aptamers are oligonucleotides that display affinity for target molecules such as proteins, small molecules, nucleic acids, and whole cells, and can have applications to clinical diagnostics and therapeutics. Certain aptamers have been employed with various transduction methods to generate diagnostic tools and/or used in therapeutics for diseases such as macular degeneration and various types of cancer. Certain so-called “smart” aptamers can be generated which bind with specific equilibrium constants, kinetic parameters, and at specific temperatures.

Aptamer sequences can be developed by an evolutionary process known as Systematic Evolution of Ligands by Exponential Enrichment, or SELEX, which can be labor-intensive.

### SUMMARY

The disclosed subject matter provides techniques for isolation, selection, and amplification of aptamers, e.g., cell-targeting aptamers.

In certain embodiments, techniques for selecting and isolating cell-targeting aptamers are provided. In certain examples, cells can be cultured on a bottom portion of a selection chamber. A sample can be introduced into the selection chamber. The sample can include cell-targeting oligomers that bind to the cultured cells. Unbound oligomers can be removed from the selection chamber to isolate the cell-targeting oligomers. In accordance with exemplary embodiments of the disclosed subject matter, the cells can be cancer cells such as MCF-7 cells. The cell-targeting oligomer can be, for example, single-stranded DNA. The unbound oligomers can be removed by infusing a washing buffer into the selection chamber.

In accordance with some embodiments of the disclosed subject matter, the cell-targeting oligomer can be eluted and hydrodynamically transferred to an amplification chamber. Elution can be accomplished by raising the temperature in the selection chamber, e.g., by controlling a heater. Microvalves in a microchannel can be actuated to hydrodynamically transfer the cell-targeting oligomers to the amplification chamber.

In certain embodiments, primer-functionalized magnetic beads can be provided in the amplification chamber. For example, the primer-functionalized magnetic beads can be held in the amplification chamber by an external magnet. The primer-functionalized magnetic beads, e.g., streptavidin-coated magnetic beads, can be configured to capture the cell-targeting oligomers. The captured oligomers can then be amplified, e.g., by applying a polymerase chain reaction technique.

The cell-targeting oligomers can be hydrodynamically transferred from the amplification chamber to the selection chamber.

In accordance with another embodiment of the disclosed subject matter, microdevices for selecting and isolating cell-targeting oligomers are provided. The microdevice can include a selection microchamber formed in a cavity of a multilayered thin film structure. The microchamber can include cells immobilized on a bottom portion thereof. The microdevice can be made by culturing cells in the selection chamber. The cells can be cancer cells such as MCF-7 cells.

In accordance with embodiments of the disclosed subject matter, the microdevice can include a heater such as a resistive heater. The heater can be serpentine-shaped. The microdevice can also include a temperature sensor.

The microdevice can include an amplification chamber connected to the selection chamber via a microchannel. The microchannel can include one or more microvalves configured to hydrodynamically transfer cell-targeting oligomers from the selection chamber to the amplification chamber. For example, the microdevice can include a pneumatic control channel configured to actuate the one or more microvalves. The pneumatic control channel can include an oil-filled channel.

The amplification chamber can include a plurality of primer-functionalized magnetic beads such as streptavidin-coated beads. The beads can be held in the amplification chamber by an external magnet. A resistive heater can be located under the amplification chamber.

The microdevice can also include an additional microchannel including one or more microvalves configured to hydrodynamically transfer cell-targeting oligomers from the amplification chamber to the selection chamber.

In certain embodiments, techniques for isolating and amplifying oligomers using a selection chamber and an amplification chamber are provided. A first sample including oligomers can be introduced into the selection chamber, and the oligomers can be isolated. The oligomers can then be hydrodynamically transferred from the selection chamber to the amplification chamber. The oligomers can then be amplified in the amplification chamber, e.g., by applying a PCR technique. The oligomers can then be transferred from the amplification chamber to the selection chamber.

In accordance with other embodiments of the disclosed subject matter, techniques for isolating and amplifying an aptamer using a selection chamber and an amplification chamber can include introducing a first sample including an oligomers into the selection chamber, isolating the oligomers, transferring the oligomers from the selection chamber to the amplification chamber, amplifying the oligomers in the amplification chamber, and hydrodynamically transferring the oligomers from the amplification chamber to the selection chamber.

The selection chamber can include immobilized targets for selecting the oligomers. The immobilized targets can be, for example, cultured cells such as cancer cells. In accordance with other embodiments, the immobilized targets can be, for example, microbeads, e.g., IgE-functionalized microbeads. The microbeads can be retained in the selection chamber by a weir structure.

The oligomers can be transferred from the selection chamber to the amplification chamber via a first microchannel. For example, the oligomers can be hydrodynamically transferred from the selection chamber to the amplification chamber, e.g., using one or more microvalves. In accordance with other embodiments, the oligomers can be transferred from the selection chamber to the amplification chamber via electrophoresis.

In certain embodiments, microdevices for selecting and isolating cell-targeting oligomers are provided. A microdevice in accordance with an exemplary embodiment of the disclosed subject matter can include a selection microchamber, an amplification microchamber, a first microchannel between the selection chamber and the amplification chamber configured to transfer oligomers from the selection chamber to the amplification chamber, and a second microchannel between the selection chamber and the amplification chamber configured to transfer oligomers from the amplification chamber to the selection chamber. At least one of the first microchannel and the second microchannel can include one or more microvalves.

The selection chamber can include immobilized targets. For example, the immobilized targets can be cultured cells. In accordance with other embodiments, the immobilized targets can be microbeads retained on a weir structure. A heater and a temperature sensor can be positioned below the selection chamber.

One or both of the first microchannel and the second microchannel can include one or more microvalves configured to hydrodynamically transfer the oligomers between the selection chamber and the amplification chamber. The microdevice can include a pneumatic control channel configured to actuate the one or more microvalves. The pneumatic control channel can be, for example, an oil-filled channel.

In accordance with some embodiments of the disclosed subject matter, the amplification chamber can include primer-functionalized microbeads. An external magnet can be positioned to hold the microbeads in the amplification chamber.

In accordance with an exemplary embodiment of the disclosed subject matter, the second microchannel can include one or more microvalves configured to hydrodynamically transfer the oligomers from the amplification chamber to the selection chamber. The microdevice can include a pneumatic control channel configured to actuate the one or more microvalves. The pneumatic control channel can be, for example, an oil-filled channel. The first microchannel can include an agarose gel for transferring oligomers via electrophoresis.

The disclosed subject matter also provides microdevices, and fabrication methods thereof, for implementing the techniques described above.

### BRIEF DESCRIPTION OF THE DRAWINGS

FIG. 1 is a flowchart of an exemplary embodiment of a process for selecting and isolating cell-targeting aptamers in accordance with the disclosed subject matter.

FIG. 2 is a flowchart of another exemplary embodiment of a process for isolating and amplifying aptamers in accordance with the disclosed subject matter.

FIG. 3 is a flowchart of another exemplary embodiment of a method for isolating and amplifying aptamers in accordance with the disclosed subject matter.

FIG. 4 is a top view of an exemplary embodiment of a microfluidic device for aptamer development in accordance with the disclosed subject matter.

FIG. 5 is a cross-sectional view of the microfluidic device of FIG. 4 along line a-a in accordance with the disclosed subject matter.

FIG. 6 is a top view of another embodiment of a microfluidic device for aptamer development in accordance with the disclosed subject matter.

FIGS. 7A-7B is a cross-section view of the microfluidic device of FIG. 6 in accordance with the disclosed subject matter. FIG. 7A shows a cross-section view along the line a-a. FIG. 7B shows a cross-section view along the line b-b.

FIGS. 8A-8H illustrates an exemplary embodiment of a method for fabricating a microdevice for aptamer development in accordance with the disclosed subject matter.

FIG. 9 is a top view of an exemplary embodiment of a microfluidic device in accordance with the disclosed subject matter.

FIG. 10A shows an exemplary system for microfluidic cell-targeting aptamer development in accordance with the disclosed subject matter. FIGS. 10B through 10M are an illustrated flow chart of an exemplary method for cell-targeting aptamer selection and amplification in accordance with the disclosed subject matter. FIG. 10B shows cell culturing on a chip. FIG. 10C shows the introduction of streptavidin magnetic beads with surface immobilized reverse primers. FIG. 10D shows washing of cells using D-PBS. FIG. 10E shows infusion of random ssDNA library for selection and removal of weakly bound ssDNA by multiple washes. FIG. 10F shows thermal elution, hydrodynamic transfer, and capture of strongly bound ssDNA. FIG. 10G shows introduction of PCR reagent and thermal cycling. FIG. 10H shows removal of cells and rinsing of selection chamber. FIG. 10I shows cell culturing on chip. FIG. 10J shows washing cells using C-PBS. FIG. 10K shows thermal release of amplified ssDNA from bead surface and transfer back to the selection chamber for isolation of strongly bound ssDNA. FIG. 10L shows removal of used streptavidin beads. FIG. 10M shows introduction of new streptavidin magnetic beads with surface immobilized reverse primers, and repetition of the process from 10F to 10M.

FIG. 11 is a graph of the time-resolved tracking of the temperatures inside the buffer-filled amplification during thermal cycling in accordance with an exemplary embodiment of the disclosed subject matter.

FIG. 12A shows a phase contract image of cells cultured in the selection chamber for 5 hours in accordance with an exemplary embodiment of the disclosed subject matter. FIG. 12B shows a polyacrimide gel gel electropherogram of amplified eluents obtained during a selection process in accordance with the disclosed subject matter.

FIG. 12C is a bar graph indicating band intensities for lanes W1-NC. Lane W1 corresponds to Wash 1. Lane W3 corresponds to wash 3. Lane W5 corresponds to Wash 5. Lane W7 corresponds to Wash 7. Lane W9 corresponds to Wash 9. Lane E1 corresponds to thermal elution at 1 μL/min and 60° C. Lane E3 corresponds to thermal elution at 10 μL/min and 60° C. Lane NC corresponds to negative control (no template).

FIGS. 13A-13D shows verification of bead-based PCR and ssDNA elution in accordance with an exemplary embodiment of the disclosed subject matter. FIG. 13A shows a fluorescent image of beads before PCR. FIG. 13B shows a fluorescent image of beads after 25 cycles of PCR. FIG. 13C shows a fluorescent image of beads after thermally induced ssDNA elution. FIG. 13D is a bar graph depicting the fluorescent intensities of the beads.

FIG. 14A shows an exemplary embodiment of a schematic of three-round, closed-loop cell-targeting ssDNA generation in accordance with the disclosed subject matter. FIG. 14B is a polyacrimide gel electropherogram of amplified eluents obtained during the three hour selection processes in accordance with an exemplary embodiment of the disclosed subject matter. FIG. 14C is a bar graph indicating band intensities for lanes W11-NC. Lane W11 corresponds to Round 1, Wash 1. Lane W19 corresponds to Round 1, Wash 9. Lane W21 corresponds to Round 2, Wash 1. Lane W29 corresponds to Round 2, Wash 9. Lane W31 corresponds to Round 3, Wash 1. Lane W39 corresponds to Round 3, Wash 9. Lane E corresponds to thermal elution. Lane NC corresponds to negative control (no template).

FIG. 15 is a bar graph of fluorescence intensity of cells incubated with isolated ssDNA and randomized ssDNA in accordance with an exemplary embodiment of the disclosed subject matter.

FIGS. 16A-16C illustrates an exemplary embodiment of a microdevice in accordance with the disclosed subject matter. FIG. 16A is a top view of the microdevice. FIG. 16B is a cross-sectional view along the line a-a in FIG. 16A. FIG. 16C is a cross-sectional view along the line b-b in FIG. 16A.

FIG. 17A is a gel electropherogram of amplified eluents obtained during a selection process in accordance with an exemplary embodiment of the disclosed subject matter. FIG. 17B is a bar graph depicting intensities of lanes W1 through E.

FIG. 18A is a gel electropherogram of amplification camber volumes collected at 5 minute intervals in accordance with an exemplary embodiment of the disclosed subject matter. FIG. 18B is a line graph depicting the fluorescent intensity.

FIGS. 19A-19D shows fluorescent images of beads before (FIG. 19A) and after (FIG. 19B) 20 cycles of PCR, as well as after 95° C. thermally induced ssDNA elution. FIG. 19D is a bar graph depicting the fluorescent intensities.

FIG. 20A is a gel electropherogram of amplified eluents obtained during closed loop selection and amplification in accordance with an exemplary embodiment of the disclosed subject matter. FIG. 20B is a bar graph depicting intensities of lanes W11 through W29.

FIG. 21 is a top view of an exemplary embodiment of a microfluidic device in accordance with the disclosed subject matter.

FIG. 22A is a gel electropherogram of amplified eluents during closed loop selection and amplification in accordance with an exemplary embodiment of the disclosed subject matter. FIG. 22B is a bar graph depicting intensities, representing the amount of ssDNA in the eluents, of lanes W11 through W49.

FIG. 23 is a line graph of fluorescence based binding affinity measurements of enriched pool towards IgE-functionalized microbeads. Error bars represent standard deviations from triplicate measurements.

FIG. 24 is a line graph of fluorescence based binding affinity measurements of randomized library used to initiate SELEX towards IgE-functionalized microbeads.

### DETAILED DESCRIPTION

The disclosed subject matter provides techniques for isolation, selection, and amplification of aptamers on a microchip. More specifically, the disclosed subject matter provides for MEMS-based microdevice platforms and associated methods for isolating and enriching aptamers for research, diagnostic, therapeutic, and other applications.

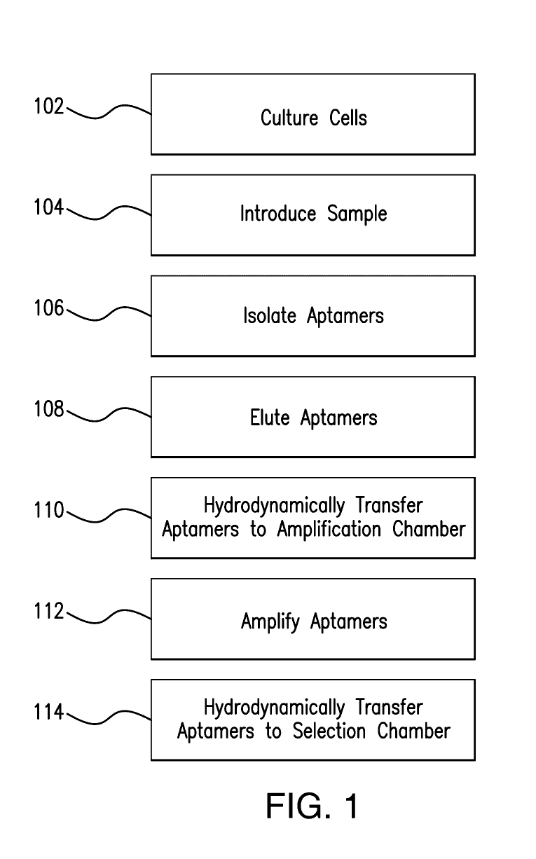

In one aspect, the presently disclosed subject matter provides a method for selecting and isolating cell-targeting aptamers. Referring to FIG. 1, the method can include culturing cells in a selection chamber (at 102). The cells can be cultured on a bottom portion of the selection chamber and can form a layer of cells that is immobilized on the bottom portion of the selection chamber. Direct cell culturing can avoid the need for chemical modification of cells (which can be needed for bead-based immobilization). Chemical modification can negatively influence the viability of cells. Direct cell culturing can also reduce the amount of stress on the cells. Stress on the cells can potentially compromise the expression of cell membrane proteins, to which cell-specific aptamers can bind.

The cells can be, for example, cancer cells. In accordance with other embodiments of the disclosed subject matter, the cells can be healthy cells. Cancer cells can be, for example, MCF-7 cells

With further reference to FIG. 1, a sample can be introduced into the isolation chamber (at 104). The sample can include cell-targeting oligomers and non-cell-targeting oligomers. As used herein, the term “non-cell-targeting oligomers” refers to oligomers that do not target the cells used for selection (i.e., the cells that immobilized in the selection chamber). However, non-cell-targeting oligomers can target other cells. The sample can be, for example, a random single-strand DNA (ssDNA) library.

Cell-targeting oligomers can include, but are not limited to, oligonucleotides between about 12 and 80 nucleotides in length. The cell-targeting oligomers can recognize a cell by specific affinity binding. Upon introduction of the sample, cell-targeting oligomers can strongly bind to the cells in the selection chamber, while non-cell-targeting oligomers can be unbound or weakly bound to the cells.

The cell-targeting oligomers can then be isolated by removing the non-cell-targeting oligomers from the selection chamber while the cell-targeting oligomers remain bound to the cells (at 106). For example, weakly bound ssDNA can be removed by washing using a washing buffer such as, for example, D-PBS. Multiple washes can be performed.

The cell-targeting oligomers can then be eluted to break the bond between the cell-targeting oligomers and the cells (at 108). For example, the cell-targeting oligomers can be eluting by adjusting the temperature of the selection chamber. For example, the selection chamber can be set at a first temperature T₁ for binding of the cell-targeting aptamer, and after the removal of the unbound DNAs and other impurities, the temperature of the second chamber can be changed, e.g., raised to T₂ which is higher than T₁ such that the conformal structure of the aptamer is disrupted, thereby releasing the cell-targeting aptamer from the cell.

The temperature control can be achieved by integrated microheaters and temperature sensors associated with the selection chamber. For example, a resistive heater and temperature sensor can be provided under the selection chamber. The microheater and the temperature sensor can be, for example, serpentine-shaped.

For certain oligomers, release temperature T₂ can be lower than the capture temperature T₁. In such cases, the lower temperature T₂ can be achieved by thermoelectric cooling, e.g., by Peltier elements incorporated in the microdevice. Alternatively, the oligomers bound to the cell can also be released using a reagent, such as an alkali solution.

With further reference to FIG. 1, the cell-targeting oligomers can be hydrodynamically transferred from the selection chamber to an amplification chamber (at 110). For example, the cell-targeting oligomers can be transported to the amplification chamber via a microchannel. The cell-targeting oligomers can be transferred using one or more microvalves. The microvalves can be, for example, elastomeric microvalves. For example, the microvalves can be constructed using SU-8. The microvalves can be actuated by a pressure-driven, oil-filled channel. The channel can be located above the microchannel between the selection chamber and the amplification channel. Hydrodynamic transfer can prevent application of an electric field (e.g., as required in electrophoresis). Exposure to high electrical fields can irreversibly damage cells.

After the cell-targeting oligomers are transferred from the selection chamber to the amplification chamber, the cells can be removed from the selection chamber. The selection chamber can be washed, e.g., using a buffer, and cell culturing can be re-started in the selection chamber.

Referring again to FIG. 1, the cell-targeting oligomers can be amplified in the amplification chamber (at 112). The amplification chamber can include primer-functionalized microbeads such as magnetic beads. For example, the magnetic beads can be polymer beads coated with streptavidin, which is known to have extraordinarily high affinity for biotin. The primer (e.g., a reverse primer) can be biotin-functionalized and immobilized onto the surface of the beads. The magnetic beads can be held in the amplification chamber by an external magnet. For example, the magnet can be placed below a bottom portion of the amplification chamber. When the sample including the cell-targeting oligomers is introduced into the amplification chamber (e.g,. via the microchannel), the cell-targeting oligomers can hybridize to the bead-immobilized primers due to molecular recognition (e.g., Watson-Crick type base pairing). Other molecules in the sample, such as non-cell-targeting oligomers, molecules, cells, small molecules, and the like, are less likely to bind with the primers.

A polymerase chain reaction (PCR) technique can be applied to amplify the cell-targeting oligomers. Using the bead-immobilized primer and PCR reagents (including e.g., Taq polymerase, deoxynucleotide triphosphates, and buffer), a complementary DNA can be produced based on the target DNA, which together with the target DNA forms a double-stranded DNA (ds-DNA) tethered on the beads. Such ds-DNA can be denatured (or melted) at an elevated temperature, e.g., about 95° C., to separate the target DNA from the complementary DNA. A second primer, e.g., a forward primer, can be annealed onto the complementary DNA (e.g., at the free end of the complementary DNA) at a lowered temperature, e.g., at 50-62° C. Thereafter, using the complementary DNA as a template, the second primer, and the PCR reagents, another copy of the target DNA can be produced, at a suitable chain extension temperature, e.g., about 72° C. Repeating the above temperature cycles (melting, annealing, and extension) can result in amplification of the target DNA, i.e., generation of exponentially increasing duplicate copies of the target DNA.

The untethered second primer can be labeled with a spectroscopically detectable tag (e.g., a fluorophore). In such a case, the result of the amplification after a number of PCR cycles can be fluorophore-labeled target DNA and unlabeled, bead-tethered complementary strands. Such labeled target DNA can be isolated for detection by fluorescent spectroscopy.

With further reference to FIG. 1, the cell-targeting aptamer can be hydrodynamically transferred from the amplification chamber to the selection chamber (at 114). For example, the cell-targeting oligomers can be transported from the amplification chamber to the selection channel via a second microchannel. The cell-targeting oligomers can be transferred using one or more microvalves. The microvalves can be, for example, elastomeric microvalves. For example, the micorvalves can be constructed using SU-8. The microvalves can be actuated by a pressure-driven, oil-filled channel. The channel can be located above the microchannel between the selection chamber and the amplification channel.

After the cell-targeting oligomers are transferred to the selection chamber, the used streptavidin beads can be removed from the amplification chamber. In accordance with certain embodiments of the disclosed subject matter, the external magnet can be removed and/or turned off. New streptavidin beads can be introduced into the amplification chamber and held in place (e.g., by replacing and/or turning on the external magnet).

In accordance with certain embodiments of the disclosed subject matter, the selection and amplification process can be repeated one or more additional times.

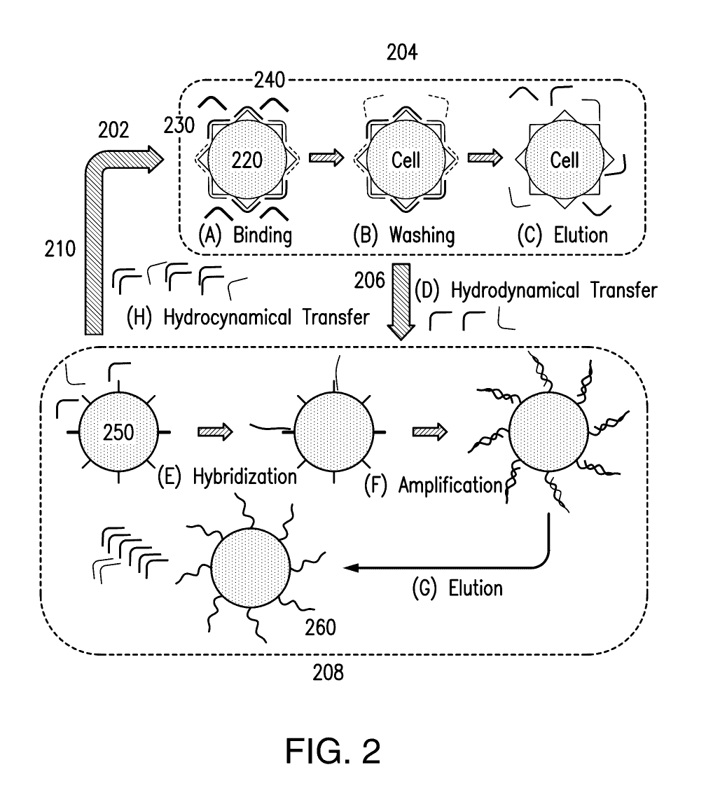

In another aspect, the disclosed subject matter provides a method for isolating and amplifying an aptamer. Referring to FIG. 2, which shows a conceptual flow chart in accordance with one embodiment of the disclosed subject matter, the method can include introducing a first sample comprising oligomers (e.g., a cell-targeting aptamer) into a selection chamber (at 202). The method can further include isolating aptamers from the oligomer libraries (204). Isolating the aptamers can include allowing the cell-targeting oligomers 230 to bind to the cell 220, while certain non-cell-targeting oligomers 240 do not bind or weakly bind to the cell 220 (at 204A). Isolating the oligomers can further include washing (at 204B), for example, by introducing a washing buffer one or more times. Washing can remove at least some of the non-cell-targeting oligomers 240 while the cell-targeting oligomers 230 remain bound to cell 220. The cell-targeting oligomers 230 can then be eluted (at 204C) such that they are no longer bound to the cell 220.

With further reference to FIG. 2, the method can also include hydrodynamically transferring the oligomers from the selection chamber to an amplification chamber (at 206). For example, the cell-targeting oligomers can be transported to the amplification chamber via a microchannel. The cell-targeting oligomers can be transferred using one or more microvalves. The microvalves can be, for example, elastomeric microvalves. For example, the microvalves can be constructed using SU-8. The microvalves can be actuated by a pressure-driven, oil-filled channel. The channel can be located above the microchannel between the selection chamber and the amplification channel. Hydrodynamic transfer can prevent application of an electric field (e.g., as required in electrophoresis). Exposure to high electrical fields can irreversibly damage cells.

Referring again to FIG. 2, the method can further include amplifying the oligomers (at 208). Amplification can include allowing hybridization of the oligomers 230 on magnetic beads 250 held in the amplification chamber (at 208E). Amplification can also include amplifying the oligomers 230 that are hybridized on magnetic beads 250, e.g., by applying polymerase chain reaction (PCR) techniques (at 208F). The aptamers 230 can then be eluted (at 208G), leaving the complementary DNA 260 bound to the magnetic beads 250.

With reference yet again to FIG. 2, the method can further include hydrodynamically transferring the aptamer from the amplification chamber to the selection chamber (at 210). For example, the cell-targeting oligomers 230 can be transported from the amplification chamber to the selection channel via a second microchannel. The cell-targeting aptamers can be transferred using one or more microvalves. The microvalves can be, for example, elastomeric microvalves. For example, the microvalves can be constructed using SU-8. The microvalves can be actuated by a pressure-driven, oil-filled channel. The channel can be located above the microchannel between the selection chamber and the amplification channel.

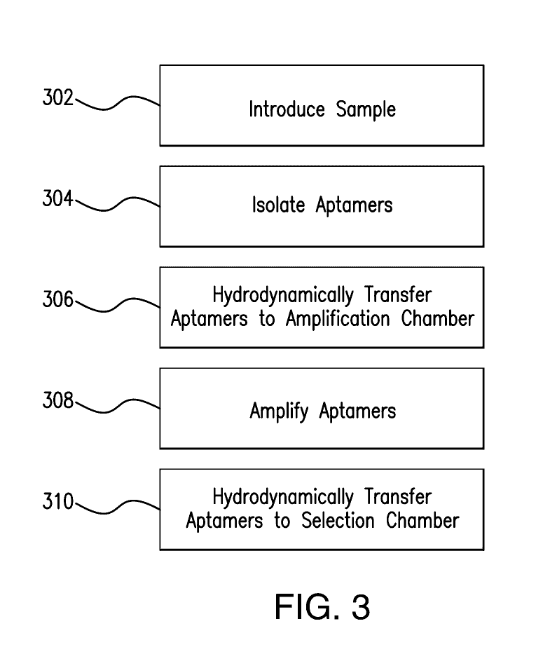

In yet another aspect, the disclosed subject matter provides a method for isolating and amplifying an aptamer. Referring to FIG. 3, the method can include introducing a first sample comprising oligomers into a selection chamber (at 302). The first sample can be, for example, a randomized ssDNA library. The selection chamber can include an immobilized target. In accordance with one embodiment of the disclosed subject matter, the immobilized target can be cultured cells. For example, the immobilized target can be a layer of cells such as cancer cells. In accordance with another embodiment, the immobilized target can include functionalized microbeads. For example, the microbeads can be Immunogolbin E-functionalized microbeads. The microbeads can be retained in the selection chamber by a weir structure. In accordance with further embodiments, the immobilized target can be immobilized metal ions, small molecules, peptides, amino acids, proteins, viruses, or bacteria.

The oligomers can then be isolated (at 304). For example, the oligomers can be allowed to strongly bind with an immobilized target, e.g., cells or IgE-functionalized microbeads. The unbound and weakly bound ssDNA can then be removed by washing, e.g., using a washing buffer such as D-PBS. The oligomers can then be eluted in preparation for transfer to the amplification chamber. In accordance with an exemplary embodiment of the disclosed subject matter, the oligomers can be thermally eluted. For example, the temperature of the selection chamber can be raised using on-chip microheaters and temperature sensors. In accordance with another embodiment, the oligomers can be chemically eluted.

With further reference to FIG. 3, the aptamer can be transferred from the selection chamber to the amplification chamber via a first microchannel (at 306). In accordance with an exemplary embodiment of the disclosed subject matter, the oligomers can be hydrodynamically transferred from the selection chamber to the amplification chamber via the first microchannel. The oligomers can be transferred using one or more microvalves. The microvalves can be, for example, elastomeric microvalves. For example, the microvalves can be constructed using SU-8. The microvalves can be actuated by a pneumatic control channel. For example, the pneumatic control channel can be a pressure-driven, oil-filled channel. The channel can be located above the microchannel between the selection chamber and the amplification channel. In accordance with other embodiments of the disclosed subject matter, the oligomers can be transferred from the selection chamber to the amplification chamber by electrophoresis.

After the oligomers are transferred from the selection chamber to the amplification chamber, the immobilized target can be removed from the selection chamber. The selection chamber can be washed, e.g., using a buffer, and a new batch of immobilized targets can be loaded in the selection chamber.

The oligomers can be amplified in the amplification chamber (at 308). The amplification chamber can include primer-functionalized microbeads. For example, the primer-functionalized microbeads can be magnetic beads such as polymer beads coated with streptavidin, which is known to have extraordinarily high affinity for biotin. The primer (e.g., a reverse primer) can be biotin-functionalized and immobilized onto the surface of the beads. The magnetic beads can be held in the amplification chamber by an external magnet. For example, the magnet can be placed below a bottom portion of the amplification chamber. When the sample including the oligomer is introduced into the amplification chamber (e.g,. via the first microchannel), the oligomers can hybridize to the bead-immobilized primers due to molecular recognition (e.g., Watson-Crick type base pairing). Other molecules in the sample, such as molecules, cells, small molecules, and the like, are less likely to bind with the primers.

A polymerase chain reaction (PCR) technique can be applied to amplify the oligomers. Using the bead-immobilized primer and PCR reagents (including e.g., Taq polymerase, deoxynucleotide triphosphates, and buffer), a complementary DNA can be produced based on the target DNA, which together with the target DNA forms a double-stranded DNA (ds-DNA) tethered on the beads. Such ds-DNA can be denatured (or melted) at an elevated temperature, e.g., about 95° C., to separate the target DNA from the complementary DNA. A second primer, e.g., a forward primer, can be annealed onto the complementary DNA (e.g., at the free end of the complementary DNA) at a lowered temperature, e.g., at 50-62° C. Thereafter, using the complementary DNA as a template, the second primer, and the PCR reagents, another copy of the target DNA can be produced, at a suitable chain extension temperature, e.g., about 72° C. Repeating the above temperature cycles (melting, annealing, and extension) can result in amplification of the target DNA, i.e., generation of exponentially increasing duplicate copies of the target DNA.

The untethered second primer can be labeled with a spectroscopically detectable tag (e.g., a fluorophore). In such a case, the result of the amplification after a number of PCR cycles can be fluorophore-labeled target DNA and unlabeled, bead-tethered complementary strands. Such labeled target DNA can be isolated for detection by fluorescent spectroscopy.

With further reference to FIG. 3, the oligomers can be hydrodynamically transferred from the amplification chamber to the selection chamber (at 310). In accordance with an exemplary embodiment of the disclosed subject matter, the oligomers can be hydrodynamically transferred from the selection chamber to the amplification chamber via a second microchannel. In another embodiment of the disclosed subject matter, the oligomers can be hydrodynamically transferred from the selection chamber to the amplification chamber via the first microchannel. The oligomers can be transferred using one or more microvalves. The microvalves can be, for example, elastomeric microvalves. For example, the microvalves can be constructed using SU-8. The microvalves can be actuated by a pneumatic control channel. For example, the pneumatic control channel can be a pressure-driven, oil-filled channel. The channel can be located above the microchannel between the selection chamber and the amplification channel. In embodiments where the oligomers can be transferred from the amplification chamber to the selection chamber using the first microchannel, the microvalves can be configured to be bi-directional.

After the oligomers are transferred to the selection chamber, the used streptavidin beads can be removed from the amplification chamber. In accordance with certain embodiments of the disclosed subject matter, the external magnet can be removed and/or turned off. New streptavidin beads can be introduced into the amplification chamber and held in place (e.g., by replacing and/or turning on the external magnet).

In accordance with certain embodiments of the disclosed subject matter, the selection and amplification process can be repeated one or more additional times.

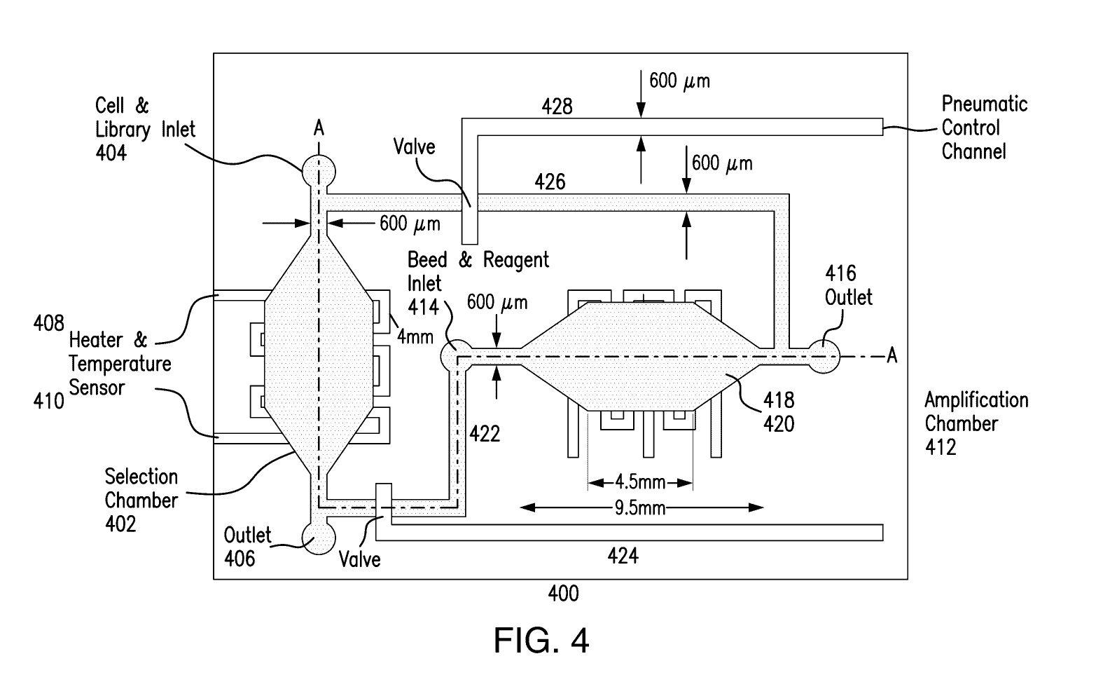

In a further aspect, the disclosed subject matter provides a microdevice for selecting and isolating cell-targeting aptamers. An exemplary embodiment of a microdevice 400 in accordance with the disclosed subject matter is illustrated in FIG. 4. As shown in FIG. 4, the microdevice 400 can include a selection chamber 402. The selection chamber can be fabricated using standard microfabrication techniques, e.g., using PDMS soft lithography to create a chamber with desired shape and dimension. For example and not limitation, the selection chamber 402 can have a semi-circular profile with a height of about 20 μm. The selection chamber can include an inlet 404 to permit introduction of samples. For example, a random ssDNA library can be introduced via the inlet 404 at the start of a systematic evolution of ligands by exponential enrichment (SELEX) process. The microdevice 400 can also include an outlet 406 to permit for disposal of waste materials. For example, the non-cell-targeting oligomers can be removed via the outlet 406 during washing.

The microdevice 400 can further include a heater 408 and a temperature sensor 410. The microheater 408 can be a resistive heater and can be formed in a serpentine shape. The temperature sensor 410 can be a resistive temperature sensor can be formed in a serpentine shape. The heater 408 and temperature sensor 410 can be used to control the temperature in the selection chamber 402 using, for example, electronic control circuitry.

The microdevice 400 can further include an amplification chamber 412. The amplification chamber 412 can include an inlet 414 and an outlet 416, and the temperature of the amplification chamber 412 can be controlled by a heater 418 and temperature sensor 420, as described in connection with the selection chamber 402.

The selection chamber 402 and the amplification chamber 412 can be coupled via a first microchannel 422. The first microchannel 422 can include one or more microvalves configured to hydrodynamically transfer oligomers from the selection chamber 402 to the amplification chamber 412. The one or more microvalves can be actuated by a first pneumatic control channel 424. The first pneumatic control channel 442 can be filled with oil.

The one or more microvalves in first microchannel 422 can further be configured to hydrodynamically transfer oligomers from the amplification chamber 412 to the selection chamber 402. Alternatively, a second microchannel 426 between the selection chamber 402 and the amplification chamber 412 can be used. The second microchannel 426 can include one or more microvalves configured to hydrodynamically transfer oligomers from the amplification chamber 412 to the selection chamber 402. The one or more microvalves in second microchannel 426 can be actuated by a second pneumatic control channel 428.

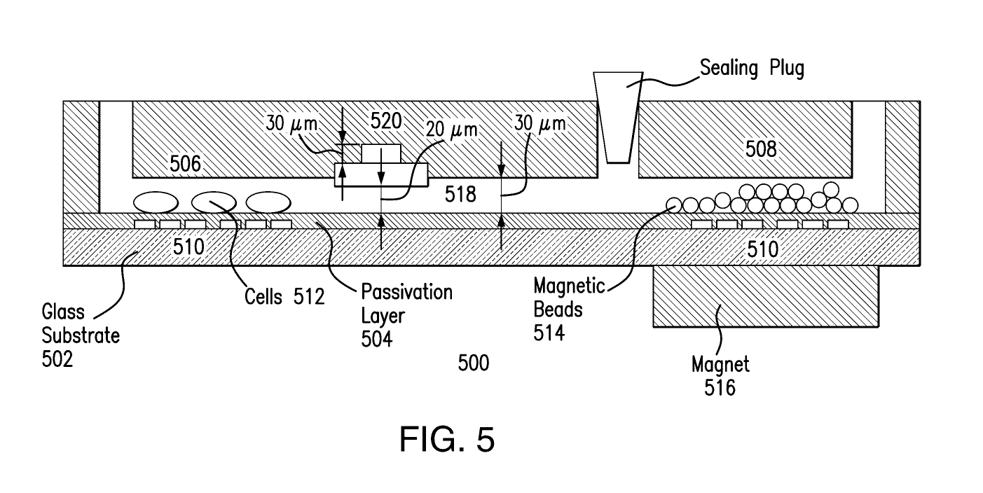

FIG. 5 illustrates a cross-sectional view of a microdevice 500 in accordance with an exemplary embodiment of the disclosed subject matter. The microdevice includes a substrate 502 such as a glass substrate. A passivation layer 504 can be situated between the substrate and the interior of the selection chamber 506 and the amplification chamber 508. Temperature control elements 510, including microheaters and temperature sensors, can be situated within the passivation layer beneath each of the selection chamber 506 and the amplification chamber 508.

The selection chamber 506 can include cultured cells 512. The cells 512 can be cultured on a bottom portion of the selection chamber 506 and can form a layer of cells that is immobilized on the bottom portion of the selection chamber. The cells can be, for example, cancer cells such as MCF-7 cells.

The amplification chamber can include primer-functionalized microbeads such as magnetic beads 514. The magnetic beads 514 can be, for example, streptavidin-coated polymer beads. The magnetic beads 514 can be held in place by an external magnet 516 positioned below the amplification chamber 508.

A microchannel 518 can connect the selection chamber 506 to the amplification chamber 508. One or more microvalves, which are not shown in FIG. 5, can be configured to hydrodynamically transfer oligomers between the selection chamber 506 and the amplification chamber 508. The one or more microvalves are actuated by a pneumatic control channel 520.

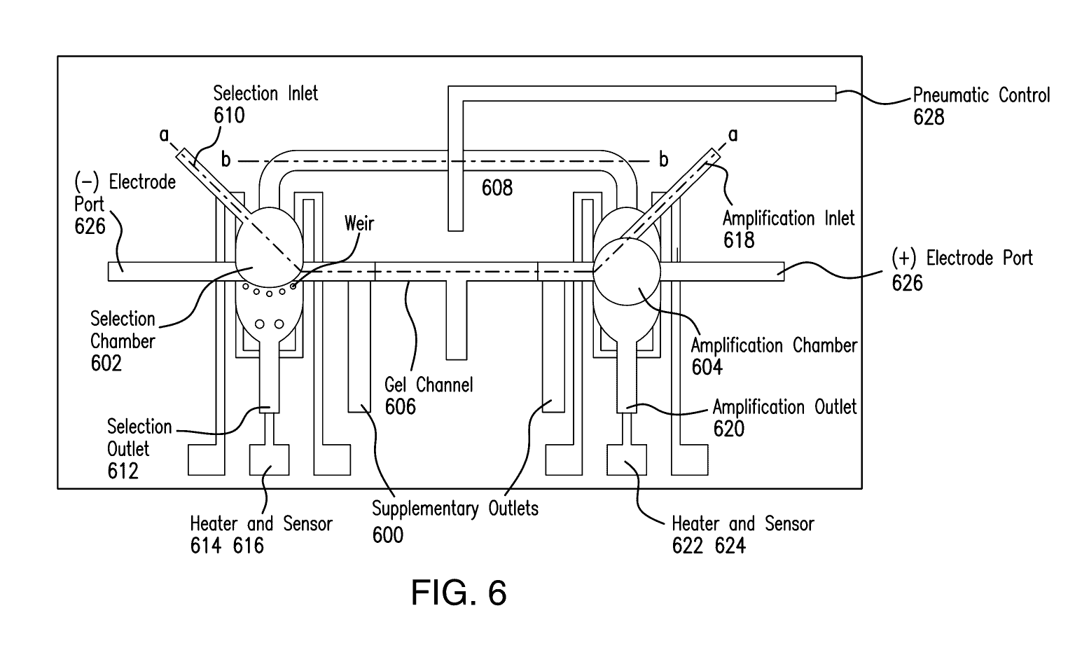

In another aspect, the disclosed subject matter provides a microdevice for isolating and amplifying an aptamer. An exemplary embodiment of a microdevice 600 in accordance with the disclosed subject matter is illustrated in FIG. 6. The microdevice can include a selection chamber 602, an amplification chamber 604, a first microchannel 606 and a second microchannel 608. The first microchannel is located between the selection chamber 602 and the amplification chamber 604 and is configured to transfer oligomers from the selection chamber 602 to the amplification chamber 604. The second microchannel is located between the selection chamber 602 and the amplification chamber 604 and is configured to transfer oligomers from the amplification chamber 604 to the selection chamber 602. At least one of the first microchannel and the second microchannel includes one or more microvalves configured to hydrodynamically transfer oligomers. In accordance with another embodiment of the disclosed subject matter, the microdevice can include only a single microchannel configured to transfer oligomers in both directions.

The selection chamber 602 and the amplification chamber 604 can be fabricated using standard microfabrication techniques, e.g., using PDMS soft lithography to create chambers with desired shape and dimension. For example and not limitation, the selection chamber 602 can have a semi-circular profile with a height of about 20 μm.

The microdevice 600 can include a selection chamber inlet 610 and a selection chamber outlet 612 for introduction and disposal of sample materials. For example, a randomized ssDNA library can be introduced via selection chamber inlet 610, while unbound and weakly bound ssDNA can be removed via the selection chamber outlet by washing. The microdevice can also include a selection chamber heater 614 and a selection chamber temperature sensor 616. The heater 614, which can be a resistive heater and be formed in a serpentine shape, and the temperature sensor 616, which can be a resistive sensor and be formed in a serpentine shape, can be located below the selection chamber 602 and can be used to control the temperature within the selection chamber 602. The microdevice can similarly include an amplification chamber inlet 618, an amplification chamber 620, an amplification chamber heater 622, and an amplification chamber temperature sensor 624.

The first microchannel 606 can be configured to transfer oligomers via electrophoresis as shown in FIG. 6. For example, the first microchannel 606 can be filled with a gel such as an agarose gel. The agarose gel can allow electrokinetically driven ssDNA migration while preventing bulk flow. First and second electrode ports 626 can be provided on opposite ends of the first microchannel 606. The first and second electrode ports 626 can be configured to receive wires such as platinum wires. The platinum wires can be coupled to an electrical circuit for generating an electric field across the first microchannel 606.

In accordance with another embodiment of the disclosed subject matter, the first microchannel 606 can be configured to hydrodynamically transfer oligomers from the selection chamber to the amplification chamber. The first microchannel can include one or more microvalves configured to hydrodynamically transfer aptamers from the selection chamber to the amplification chamber. The one or more microvalves can be actuated by a first pneumatic control channel. The first pneumatic control channel can be filled with oil.

The second microchannel 608 can be configured to hydrodynamically transfer aptamers from the amplification chamber to the selection chamber. For example, the second microchannel can include one or more microvalves configured to hydrodynamically transfer oligomers from the amplification chamber 604 to the selection chamber 602. The one or more microvalves can be actuated by a pneumatic control channel 628.

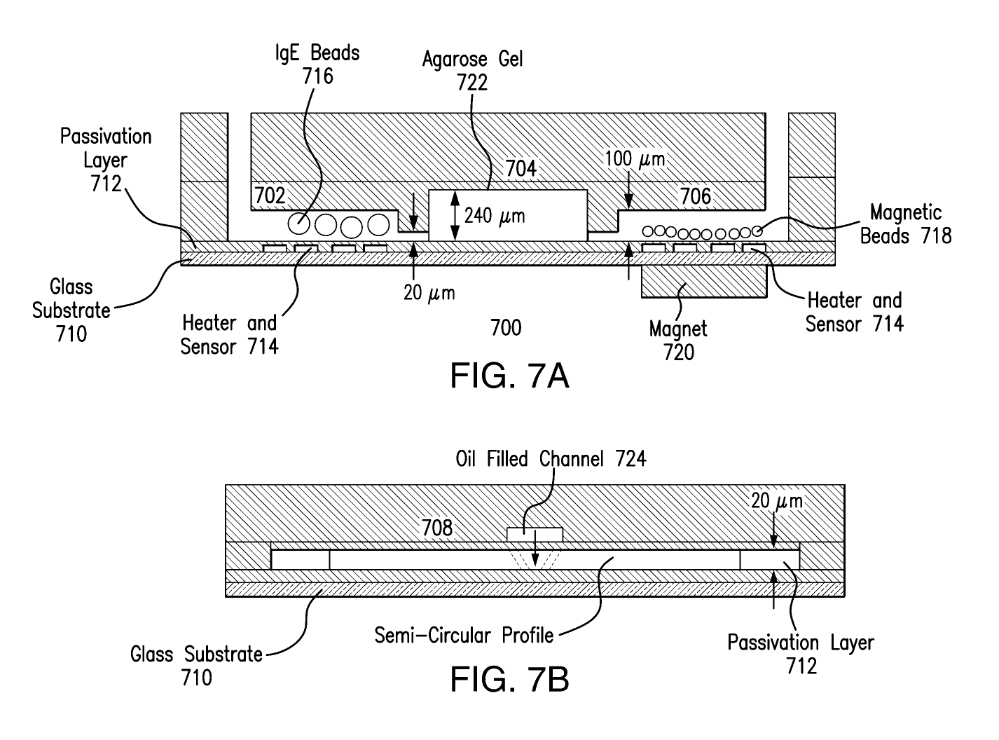

FIGS. 7A-7B illustrates a cross-sectional view of a microdevice 700 in accordance with an exemplary embodiment of the disclosed subject matter. FIG. 7A shows a cross-sectional view of microdevice 700 including a selection chamber 702, a first microchannel 704, and an amplification chamber 706. FIG. 7B shows a cross-sectional view of microdevice 700 including a second microchannel 708.

The microdevice 700 can include a substrate 710 such as a glass substrate. A passivation layer 712 can be situated between the substrate 710 an the interior of the selection chamber 702 and the amplification chamber 704. Temperature control elements 714 can be positioned below each of the selection chamber 702 and the amplification chamber 704.

The selection chamber 702 can include immobilized targets 716. For example, the immobilized targets 716 can be Immunoglobin E-functionalized microbeads, as shown in FIGS. 7A-7B. In accordance with other embodiments of the disclosed subject matter, the immobilized targets 716 can be, for example, cells (such as cancer cells cultured in the selection chamber 702), metal ions, small molecules, peptides, amino acids, proteins, viruses, and bacteria.

The amplification chamber 704 can include primer-functionalized microbeads 718. The primer-functionalized microbeads 718 can be magnetic beads such as, for example, polymer beads coated with streptavidin. The magnetic beads can be held in the amplification chamber 704 by a magnet such as an external magnet 720 positioned below the amplification chamber 704.

The first microchannel 706 can be configured to transfer oligomers from the selection chamber 702 to the amplification chamber 704 via electrophoresis. For example, as shown in FIGS. 7A-7B, the first microchannel 706 can be filled with a gel such as agarose gel 722. In accordance with another embodiment of the disclosed subject matter, the first microchannel 706 can be configured to hydrodynamically transfer oligomers from the selection chamber to the amplification chamber. The first microchannel can include one or more microvalves configured to hydrodynamically transfer oligomers from the selection chamber to the amplification chamber. The one or more microvalves can be actuated by a first pneumatic control channel. The first pneumatic control channel can be filled with oil.

With reference to FIG. 7B, the second microchannel 708 can be configured to hydrodynamically transfer oligomers from the amplification chamber 704 to the selection chamber 702. For example, the second microchannel 708 can include one or more microvalves configured to hydrodynamically transfer oligomers from the amplification chamber 704 to the selection chamber 702. The one or more microvalves can be actuated by a pneumatic control channel 724. The pneumatic control channel 724 can be located in a PDMS layer above the second microchannel 708.

Selected aptamers can be used in a variety of applications, including research, diagnostic, and therapeutic applications. For example, aptamers selected by cancer cells can be used to identify those cancer cells in blood or tissue samples. In accordance with other embodiments, aptamers can be conjugated with a toxin and can be used to induce apoptosis in a target. Aptamers can also be used to block specific antigens to inhibit cellular functions, to separate out biomolecules of interest in chromatography applications, and as affinity probes in capillary electrophoresis (CE). In connection with the latter application, aptamer-target complexes can change the electrophoretic properties and can be detected through CE.

Further details of device structure, fabrication, and operation procedures of the above-described embodiments can be found in the following Examples, which are provided for illustration purpose only and not for limitation.

### EXAMPLE 1

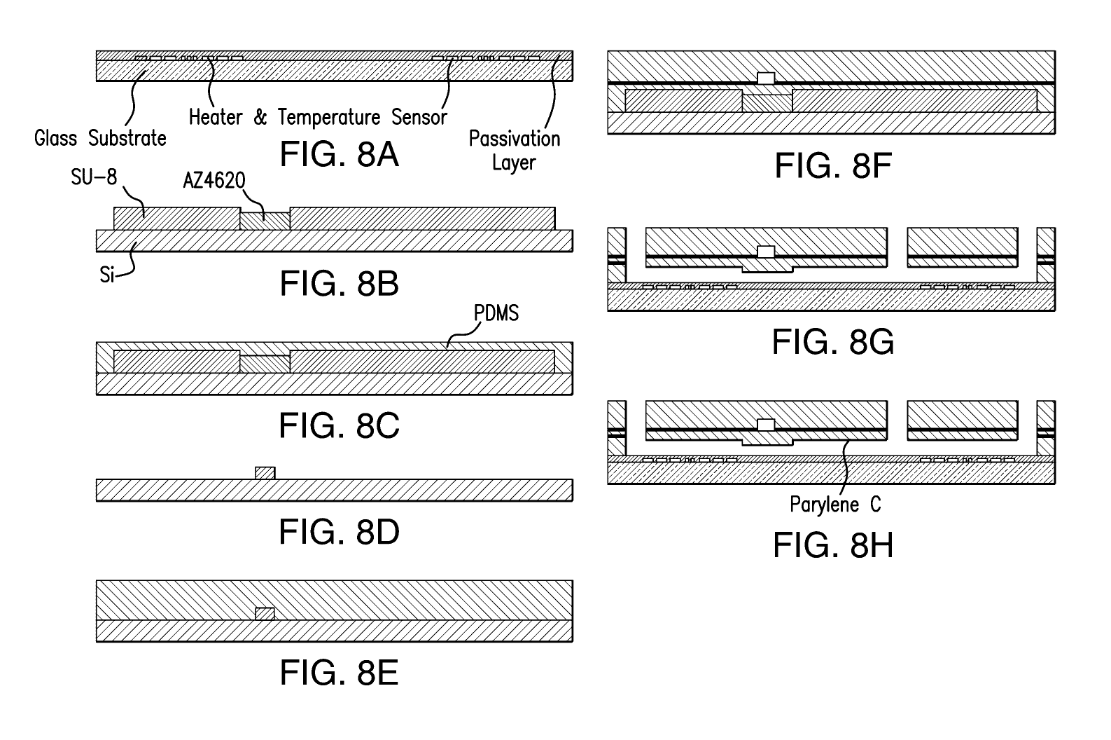

A temperature control chip was fabricated using standard microfabrication techniques. Briefly, gold (100 nm) and chrome (5 nm) thin films were thermally evaporated onto the glass substrate, and patterned by photolithography and wet etching, which resulted in resistive temperature sensors and resistive heaters. Then, 1 μm of silicon dioxide was deposited using plasma-enhanced chemical vapor deposition (PECVD) to passivate sensors and heaters, the contact regions for electrical connections to which were opened by etching the oxide layer using hydrofluoric acid (FIG. 8A). Subsequently, the microfluidic slab bearing microfluidic and pneumatic features was fabricated from PDMS using soft lithography techniques. A layer of AZ-4620 positive photoresist (20 μm, Clariant Corp. Somerville, N.J.) was spin-coated on a silicon wafer (Silicon Quest International, Inc., San Jose, Calif.), exposed to ultraviolet light through photomasks, developed, and baked to form a round-shaped flow channels that can be sealed completely. Then, a layer of SU-8 photoresist was patterned to finalize the mold defining microfluidic features (FIG. 8B). Next, a PDMS prepolymer solution (base and curing agent mixed in a 10:1 ratio) was spin-coated onto the silicon wafer, and cured on a hotplate at 72° C. for 15 min (FIG. 8C). In parallel, a layer of SU-8 photoresist was patterned on another silicon wafer to establish pneumatic controlled oil-filled valve actuation channels (FIG. 8D). Another PDMS prepolymer solution was cast onto the mold and cured on a hotplate at 72° C. for 1 hour (FIG. 8E). The resulting PDMS slab was peeled off from the mold, punched to form pneumatic inlet, and bonded to the PDMS membrane on the silicon mold bearing the microfluidic features (FIG. 8F). Afterward, the PDMS slab together with the thin PDMS membrane was peeled off, punched to establish fluidic inlets and outlets, and then bonded onto the temperature control chip irreversibly after another oxygen plasma treatment (FIG. 8G).

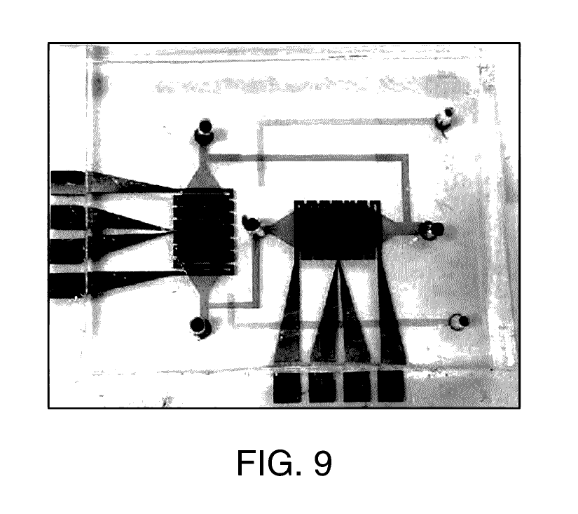

Finally, the surface of SBE microchanmber was coated with a thin layer of Parylene C via chemical vapor deposition (FIG. 8H). A fabricated device is shown in FIG. 9). The device of FIG. 9 includes a selection chamber and an amplification chamber connected by first and second microchannels controlled by first and second pneumatic control channels, along with associated heaters and temperature sensors.

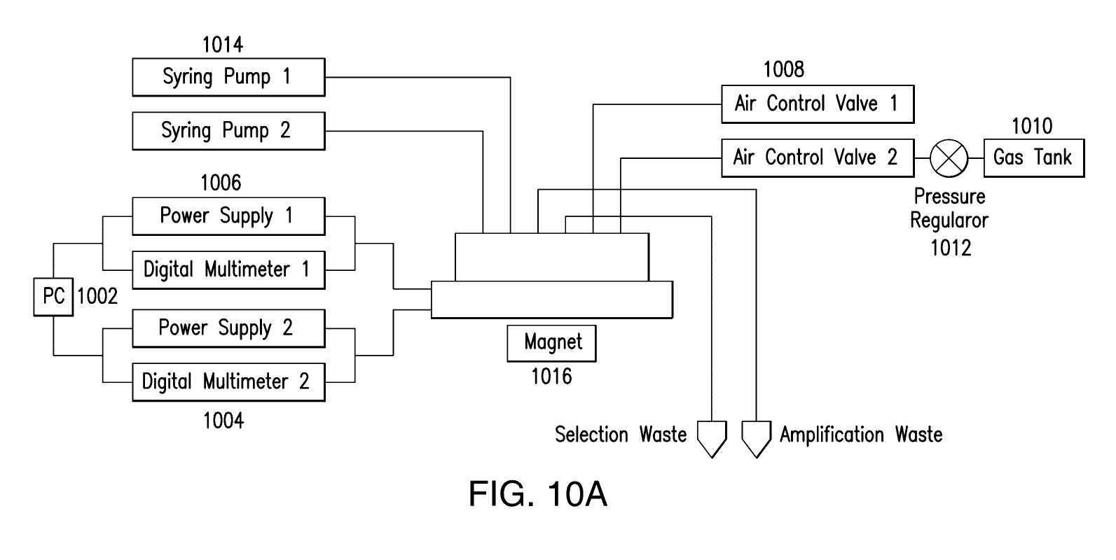

All chemicals were purchased from Sigma-Aldrich (St. Louis, Mo.) unless otherwise indicated. ssDNA random library (5′-GCC TGT TGT GAG CCT CCT GTC GAA-40N-TTG AGC GTT TAT TCT TGT CTC CC-3′ (SEQ ID NO: 1)) and primers (Forward Primer: 5′-FAM-GCC TGT TGT GAG CCT CCT GTC GAA-3′ (SEQ ID NO: 2), and Reverse Primer: 5′-dual biotin-GG GAG ACA AGA ATA AAC GCT CAA-3′ (SEQ ID NO: 3)) were synthesized and purified by Integrated DNA Technologies (Coralville, Iowa). Deoxynucleotide triphosphates (dNTPs) and GoTaq Flexi DNA polymerase were obtained from Promega Corp. (Madison, Wis.). Minimum Essential Medium (MEM), fetal bovine serum (FBS), penicillin-streptomycin (P/S, penicillin 10,000 unit/mL, streptomycin 10,000 □g/mL), Dulbecco's phosphate-buffered saline (D-PBS), 0.25% Trypsin-EDTA and streptavidin coupled magnetic beads (Dynabeads® M-270 Streptavidin) were purchased from Invitrogen (Carlsbad, Calif.). MCF-7 cell line was obtained from the American Type Culture Collection (ATCC, Manassas, Va.). Closed-loop temperature control of both selection microchamber and amplification microchamber was achieved using two groups of temperature control units with a proportional-integral-derivative (PID) algorithm implemented in a LabVIEW (National Instruments Corp., TX) program on a personal computer 1002. Each temperature control unit contained a serpentine-shaped resistive temperature sensor and a resistive heater. The resistance of two temperature sensors was measured by a digit multimeter 1004 (34410A, Agilent Technologies Inc., CA) and a digit micro-ohm meter (34420A, Agilent Technologies Inc., CA), respectively. Each resistive heater was connected to an independent, dedicated DC power supply 1006 (E3631, Agilent Technologies Inc., CA) (FIG. 10A).

Fluid control was achieved using microfabricated pressure-driven valves 1008. Two oil-filled channels, each actuated by an air control valve (6464K16, McMaster-Carr, NJ), are connected to a nitrogen gas tank 1010 (Tech Air, NY) via a pressure regulator 1012 (CONCOA North America, VA). The microfluidic device inlets are connected to a set of syringes that contain samples, buffers and reagents driven by syringe pumps 1014 (KD210P, KD Scientific, MA).

Development of aptamers in the microfluidic device starts from culturing MCF-7 cells in the selection chamber for a sufficiently long time (>4 hours) to ensure cell attachment and surface biomarker regeneration (FIG. 10B). Next, streptavidin magnetic beads with surface immobilized primers (approximately 5 pmol) are introduced and held in the amplification chamber with a magnet 1016 (FIG. 10C). Selection of ssDNA is then performed by washing the cells with D-PBS (10 μL/min FIG. 10D), infusing a random ssDNA library (100 pmol) in 20 μL binding buffer (900 mL of D-PBS+4.5 g of glucose+5 mL of 1 M MgCl2+1 g of bovine serum albumin+100 mL of FBS) through the chamber (1 μL/min, 37° C.), followed by nine washes using washing buffer (900 mL of D-PBS+4.5 g of glucose+5 mL of 1 M MgCl2+100 mL of FBS) (10 μL/min, 37° C.) for 3 min each to remove weakly bound ssDNA (FIG. 10E). Afterward, the remaining strongly bound ssDNA are thermally eluted (60° C.), hydrodynamically transferred to the amplification chamber (1 μL/min, 10 min), and captured by the surface immobilized primers (FIG. 10F). Subsequently, 2 μL of PCR reagent, including 7 pmol of forward primer, 1× GoTaq Flexi buffer, 0.5 U of GoTaq Flexi DNA polymerase, 1 nmol of dNTP and 4 nmol of MgCl2, is introduced and subjected to 30 thermal cycles of 95° C. for 15 s, 59° C. for 30 s, and 72° C. for 45 s (FIG. 10G). Cells in the selection chamber are then detached using 0.25% trypsin-EDTA, followed by a D-PBS wash (FIG. 10H). Target cells are next cultured (FIG. 10I) and washed (FIG. 10J) again. Afterward, ssDNA are released from the bead surfaces at 95° C. and are transported back to the selection chamber at 37° C. (1 μL/min) for 10 min, followed by nine washes using washing buffer (10 μL/min, 37° C.) for 3 min each to remove weakly bound ssDNA (FIG. 10K). Subsequently, streptavidin magnetic beads are removed and the amplification chamber is rinsed using D-PBS (FIG. 10L). Then, the new streptavidin magnetic beads with surface immobilized primers (approximately 5 pmol) are introduced again (FIG. 10M), and the process can be repeated (FIG. 10F).

The temperature sensor was first calibrated using an environmental test chamber (9023, Delta Design Inc., CA) maintained at a series of temperatures which are measured with high accuracy temperature reference probes (5628, Fluke Calibration, UT). The measured resistance (R) of the thin-film gold temperature sensor was observed to vary linearly with temperature (T), represented by the relationship R=R₀[1+α(T−T₀)], where R₀ is the sensor resistance at a reference temperature T₀, and α is the temperature coefficient of resistance (TCR) of the sensor.

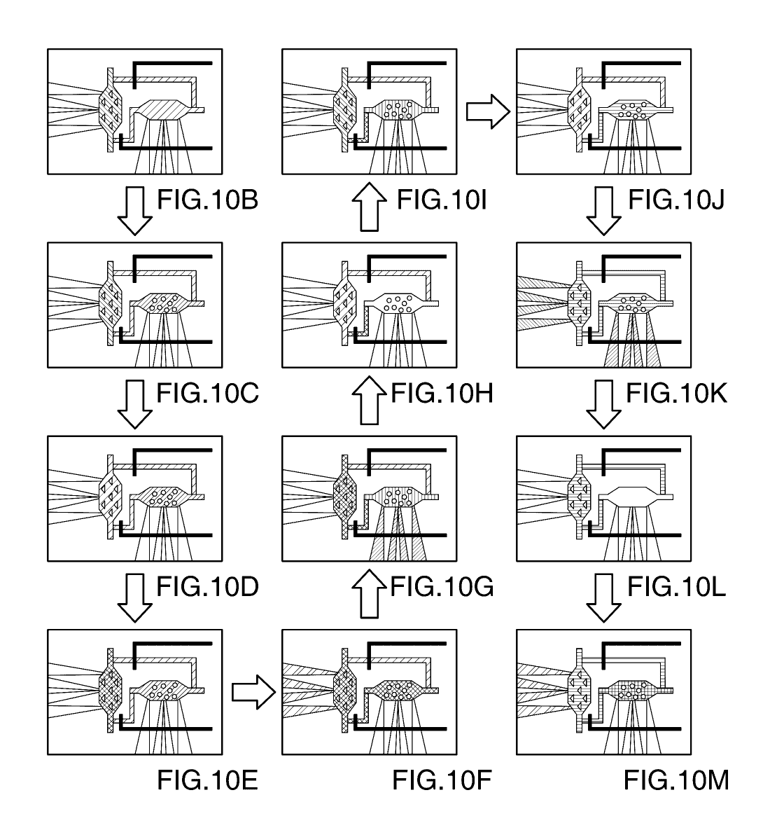

Fitting this relationship to the measurement data allowed determination of the parameter values, which were used to determine the chamber temperature from the measured sensor resistance during the selection, elution and amplification process. The temperature sensor under the amplification chamber had a measured resistance of 136.42 Ω at a reference temperature of 25.0° C. with a TCR of 3.10×10-3 1° C. The temperature control of the chamber was then characterized during thermal cycling. Time-resolved tracking of on-chip thermal cycling showed that the amplification chamber temperatures attained specified setpoints via control of the on-chip heater and off-chip fan quickly and precisely (FIG. 11).

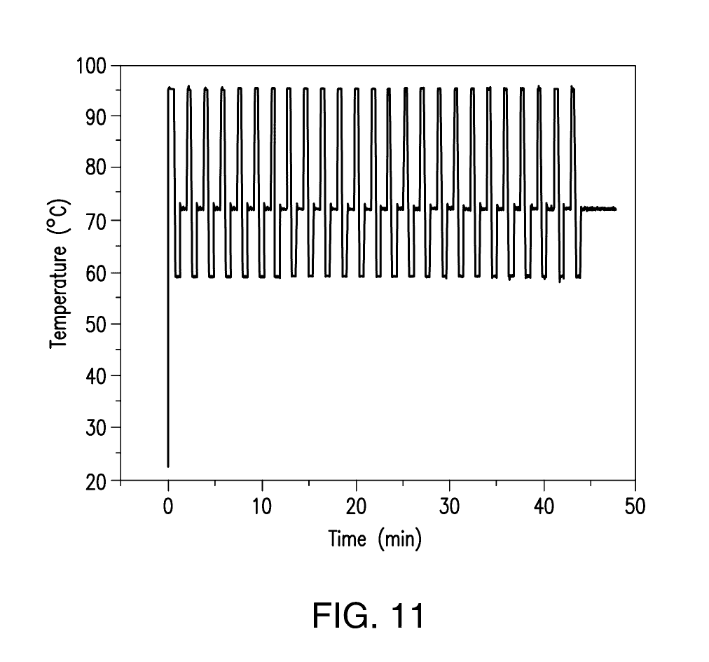

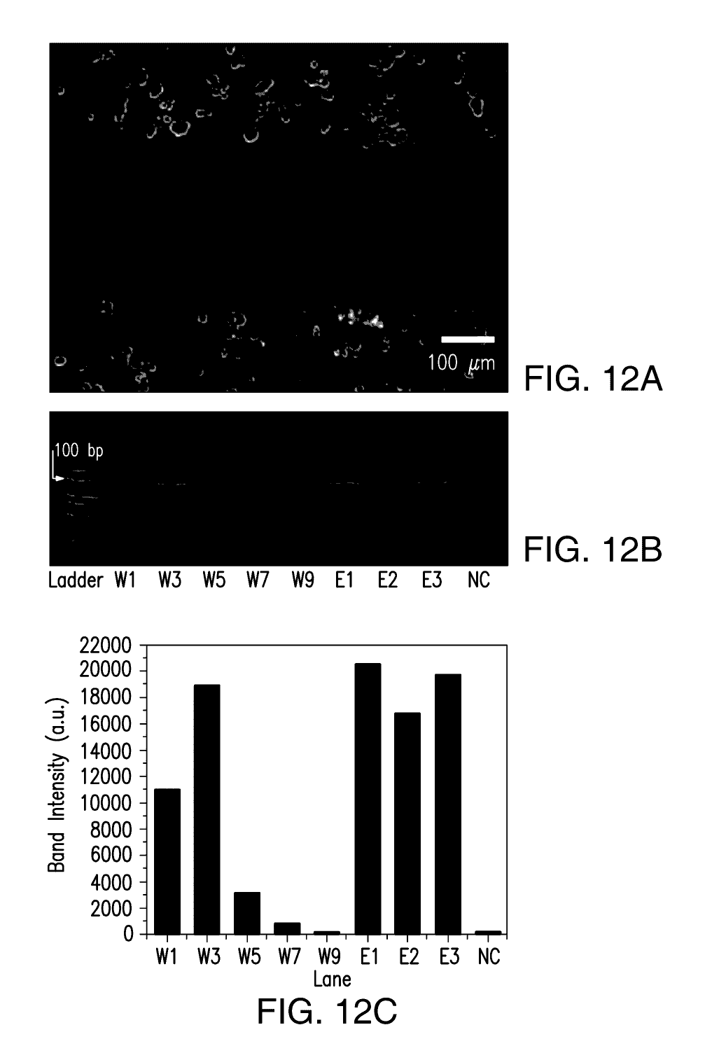

The on-chip cell culture in the selection chamber was then investigated. 2 μL of MCF-7 cell suspension at 1×107 cells/mL in complete culture media was introduced into the selection chamber, which was then kept at 37° C. in a humidified incubator containing 5% CO2 for 5 hours. The selection chamber was next rinsed using D-PBS at 10 μL/min for 1 min to remove unattached and dead cells, and a phase contract image was taken with an inverted microscope (IX81, Olympus Corp., PA) equipped with a digital camera (C8484, Hamamatsu Corp., NJ). Cells were attached well on the bottom surface (FIG. 12A), indicating the success of using this method to restrain cells in the chamber and to regenerate cell membrane proteins.

To characterize the isolation of cell-binding ssDNA from a random library, the temperature of selection chamber was kept at 37° C. for the whole procedure by using the temperature control unit located beneath. 100 pmol of ssDNA library in 20 μL binding buffer was infused into the chamber at 1 μL/min for 20 min. Then, cells were washed with 9 aliquots of washing buffer at 10 μL/min, each for 3 min, to remove undesired ssDNA. Waste from each buffer wash were collected, amplified using PCR, and analyzed using polyacrylamide gel electrophoresis (PAGE), as shown in FIG. 12B. As the cells continue to be rinsed, the amount of weakly bound ssDNA in each washing waste (identically amplified) decreases, indicated by the decreased band intensities of lanes W1 to W9 (FIG. 12C). In addition, the comparable band intensity of W9 to negative control (NC, no template in the PCR reagent) suggests that ssDNA did not exist in the waste of final washing. The low intensity of the desired band in lane W1 is caused by an unspecific amplification when using a high concentration of weakly bound ssDNA from the first wash as the template, indicated by the high fluorescence intensity above the desired band (FIG. 12B). This phenomenon can be potentially avoided by optimizing the PCR conditions when amplifying the ssDNA in the wash waste, which was not the emphasis of this work. The results indicate that the extensive washing can efficiently remove most of the weakly bound ssDNA from cell surface.

To demonstrate the thermal elution of strongly bound ssDNA after washing, the microchamber temperature was raised to 60° C. using the same temperature control unit. The cells were then rinsed with 3 aliquots of washing buffer (10 μL) at 1 μL/min, 5 μL/min and 10 μL/min, respectively. The high band intensity of lane E1 (1 μL/min), E2 (5 μL/min) and E3 (10 μL/min) indicates successful enrichment of cell-binding ssDNA (FIGS. 12B-12C). In addition, although not able to elute all the strongly bound ssDNA, rinsing cells at 1 μL/min for 10 min is sufficient to remove and collect a significant amount of cell-binding ssDNA, as suggested by the highest fluorescence intensity of the desired band at lane El (FIGS. 12B-12C). These results indicate successful enrichment and retrieval of cell-binding ssDNA.

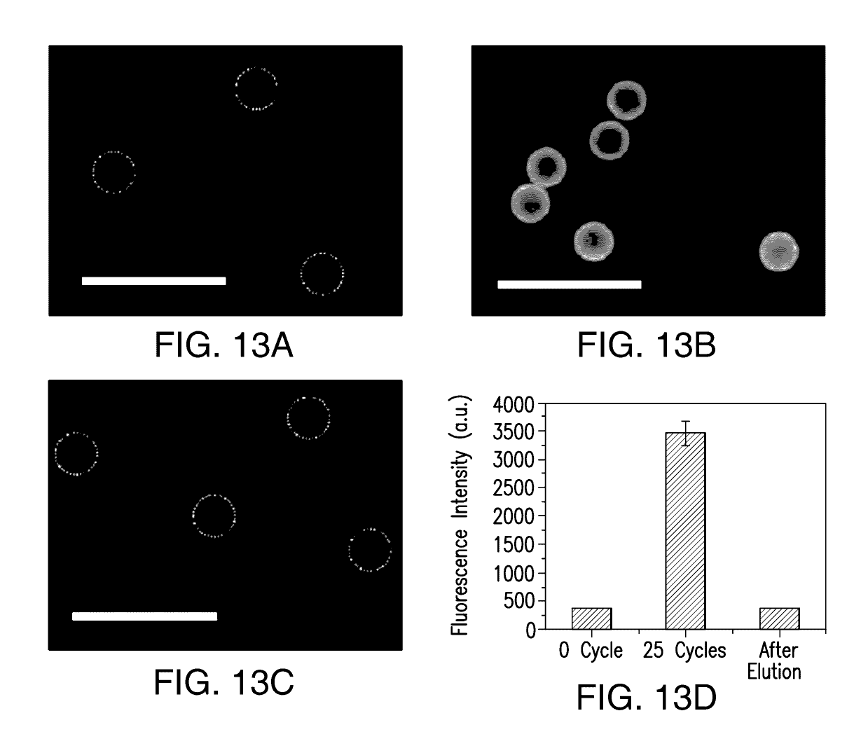

To characterize the bead-based amplification of cell-binding ssDNA, 10 μL thermal eluent was driven to the amplification chamber with primer-coated magnetic beads at 1 μL/min and room temperature for 10 min. The magnetic beads were gently stirred by the permanent magnet to achieve a better capture efficiency. Then, 2 μL of PCR reactants were introduced and thermally cycled. After washing, the fluorescent images of beads with and without cycling were taken (FIGS. 13A-13B), and fluorescence intensities of beads were then measured and compared (FIG. 13D). Following amplification, the fluorescent bead intensity was 10-fold higher, indicating efficient ssDNA amplification through bead-based PCR.

To collect the amplified cell-binding ssDNA, the magnetic beads were rinsed at 95° C. and 1 μL/min for 10 min. The rinsed beads (FIG. 13C) showed an intensity that was 10% of the pre-elution intensity, and was only 2.6% higher than that of pre-thermal cycling intensity (FIG. 13D), indicating a highly efficient ssDNA dehybridization and separation from surface immobilized complementary strands.

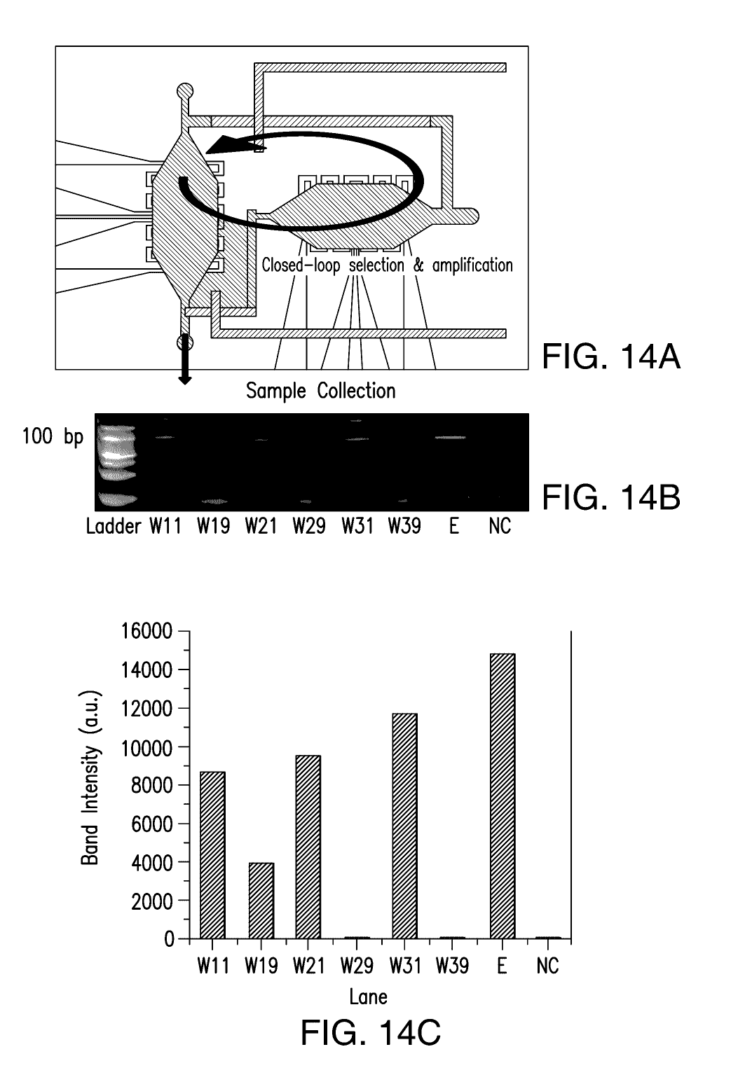

To demonstrate the feasibility of multiple-round, closed-loop cell specific aptamer generation, a three-round ssDNA selection, enrichment and amplification process was carried out. The weakly bound ssDNA of each wash in all three rounds, and the thermally eluted strongly bound ssDNA in the third round were collected from the selection chamber (FIG. 14A), amplified and analyzed with PAGE (FIG. 14B). In the first round, there was still some ssDNA in the waste of the 9th wash. However, in the second and third round, there was nearly no ssDNA in the waste of the 9th wash. This indicates that the aptamer generation process was able to successfully increase the binding affinity of ssDNA pool to the target cells after each round. In addition, the high fluorescent intensity of the thermal elution lane in the gel image (FIG. 14C) suggests that ssDNA specific to the target cells were successfully isolated, enriched and amplified.

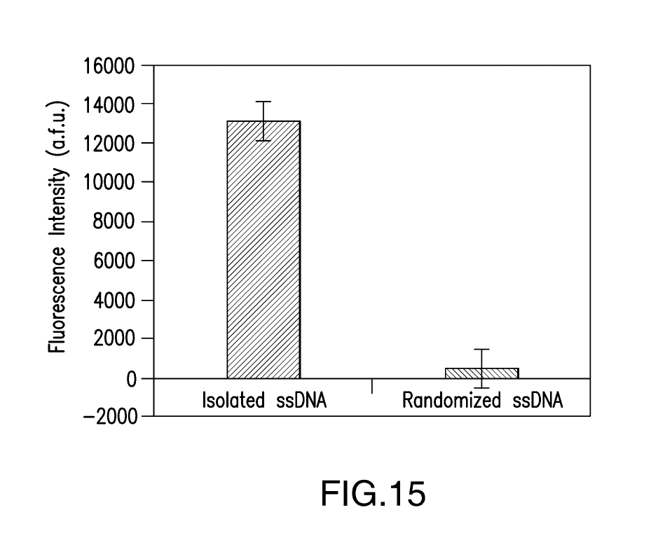

The ssDNA isolated from the microfluidic SELEX process was tested for their affinity towards MCF-7 cells using a fluorescence binding assay. The background-subtracted average fluorescence intensity of cells incubated with the enriched aptamer candidate pool was 27-fold higher than that of cells incubated with randomized ssDNA (FIG. 15), indicating that the aptamer candidates had significantly higher binding affinity to the target cells and suggesting that multi-round SELEX process using the microchip was successful.

### EXAMPLE 2

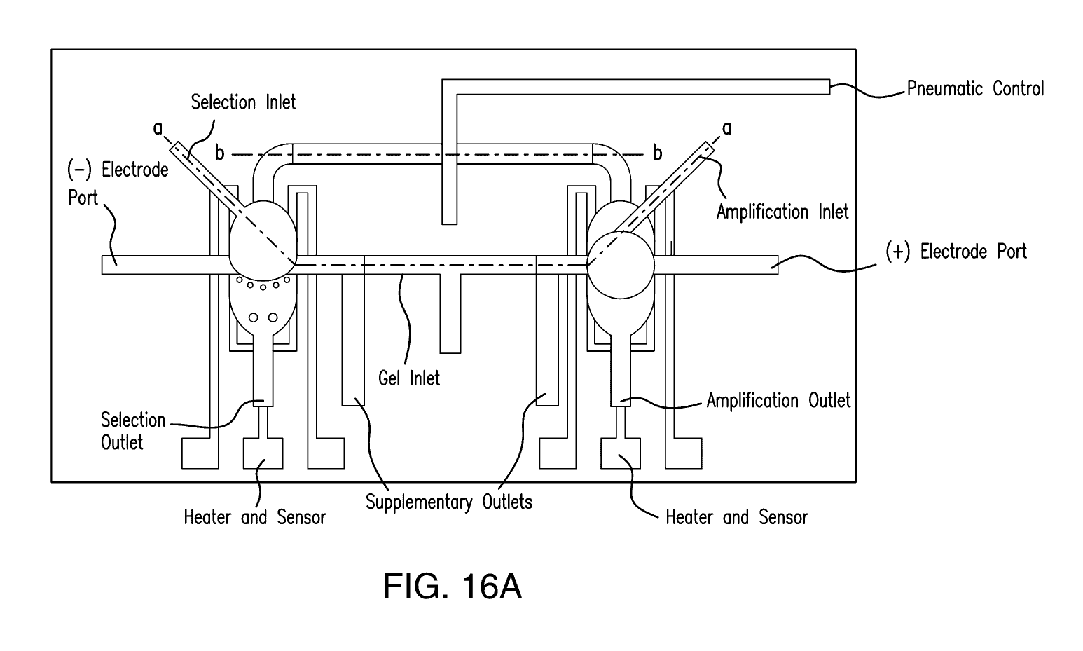

The device includes two (selection and amplification) microchambers (1.7 μL each) connected via two microchannels (FIG. 16A): one filled with gel that allows electrokinetically driven DNA migration while preventing bulk flow (FIG. 16B), the other equipped with microvalves actuated by pressurized oil from another layer of channels above (FIG. 16C).

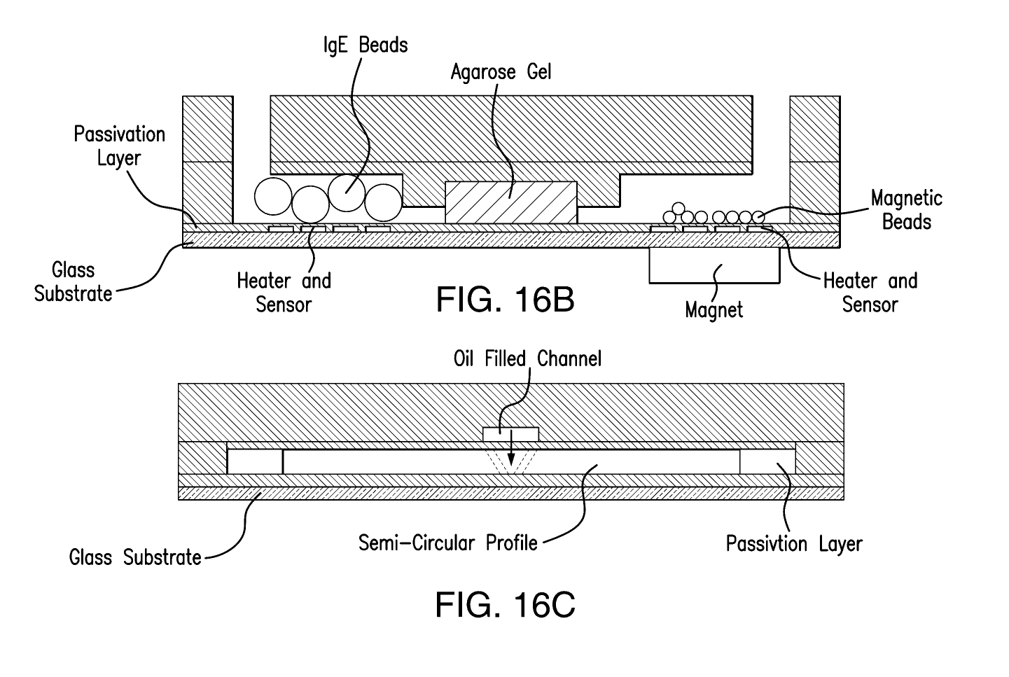

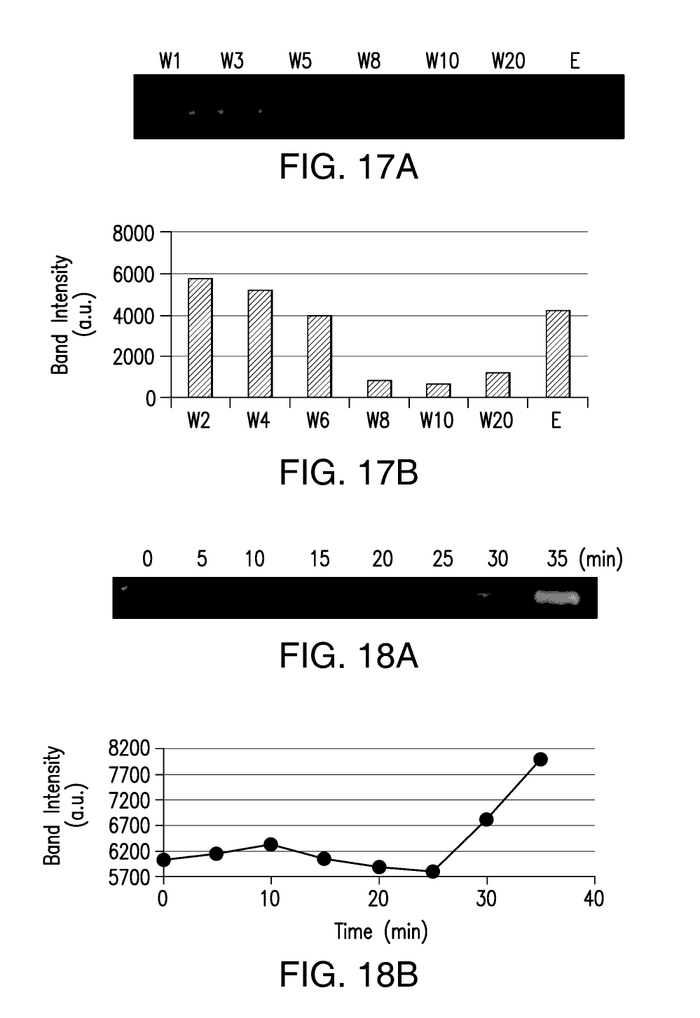

To investigate oligomer binding to IgE, amplified eluents (16 cycles via PCR) were collected during ssDNA library introduction and washing, and visualized with gel electrophoresis (FIGS. 17A-17B). The decrease in band intensity from wash 1 to wash 20, demonstrates that weakly bound ssDNA were eluted from the bead surfaces during wash, while the strong band intensity in the elution lane (E) demonstrates that ssDNA strongly bound to the bead surfaces were eluted when heating the chamber to 60 C. The electrophoretic transfer was verified by introducing an ssDNA library into the selection chamber, generating a 25 V/cm electric field with platinum-wire electrodes inserted into the electrode ports, and collecting and replacing the amplification chamber with TBE buffer every 5 minutes (FIGS. 18A-18B). The volume collected from the amplification chamber was amplified via 16 cycles of PCR and visualized with gel electrophoresis.

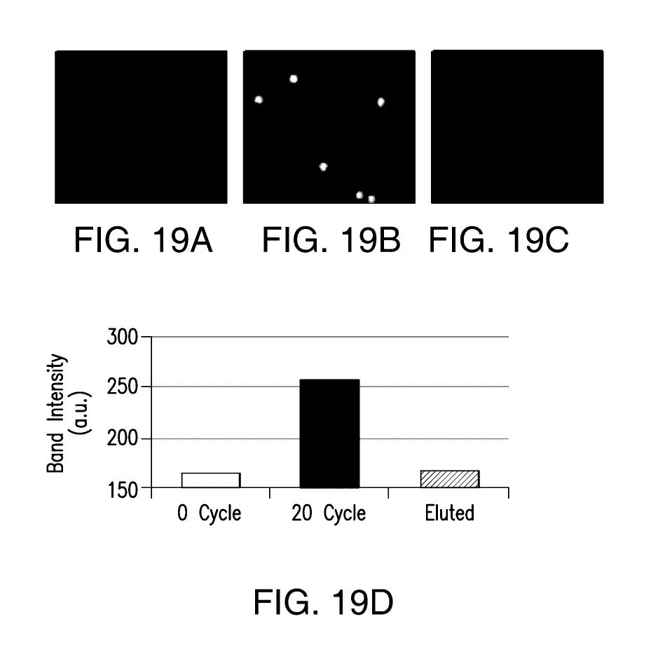

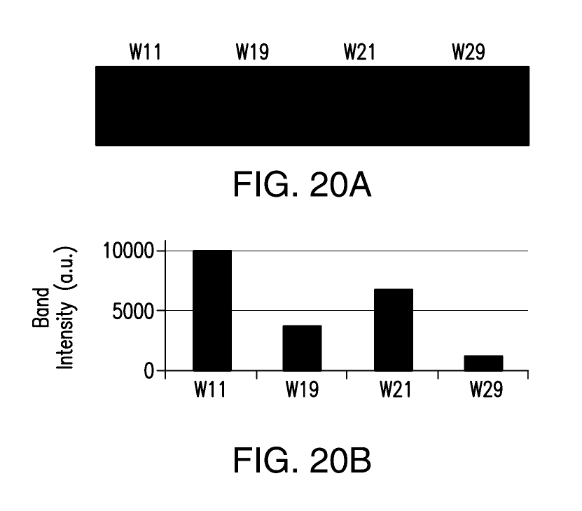

Bead based PCR was confirmed by introducing the ssDNA library into the amplification chamber containing reverse primer functionalized beads and using the integrated resistive temperature sensor and heater to perform PCR (FIGS. 19A-19D). The increase in fluorescent intensity of beads following PCR and the subsequent decrease upon heating to 95° C. indicates the successful amplification and elution, respectively, of DNA from the bead surfaces. The device was then used for isolation of affinity oligonucleotides via closed loop affinity-selection and amplification where ssDNA were selected, electrokinetically transferred, amplified, and pressure-driven back to the selection chamber for another round of selection. The decrease in band intensity from first wash (W11) to last wash (W19) of the first selection-amplification round (FIGS. 20A-20B) demonstrate successful removal of weakly bound DNA while the increase in band intensity in the effluent of the first wash of the second round (W21) indicates ssDNA were successfully amplified and transferred back to the selection chamber.

### EXAMPLE 3

The device includes two (selection and amplification) microchambers each of 1.7 uL volume. The microchambers are equipped with electrode ports for the insertion of platinum wires which generate an electric field for electrokinetics. The selection microchamber features a weir structure for capturing microbeads. The selection and amplification microchambers are connected via two microchannels (as shown in FIG. 6): one filled with agarose gel that allows electrokinetically driven ssDNA migration while preventing bulk flow (FIG. 7A), the other equipped with microvalves actuated by pressurized oil from another layer of channels above (FIG. 7B).

All chemicals were purchased from Sigma-Aldrich (St. Louis, Mo.) unless otherwise indicated. Deoxyribonucleotide triphosphates (dNTPs) and GoTaq Flexi DNA polymerase were obtained from Promega Corp. (Madison, Wis.). Randomized oligomer library (5′-GCC TGT TGT GAG CCT CCT GTC GAA-45N-TTG AGC GTT TAT TCT TGT CTC CC-3′ (SEQ ID NO: 4)) and primers (Forward Primer: 5′-FAM-GCC TGT TGT GAG CCT CCT GTC GAA-3′ (SEQ ID NO: 2), and Reverse Primer: 5′-dual biotin-GG GAG ACA AGA ATA AAC GCT CAA-3′ (SEQ ID NO: 3)) were synthesized and purified by Integrated DNA Technologies (Coralville, Iowa). Human Myeloma Immunoglubulin E (IgE) was purchased from Athens Research and Technology (Athens, Ga.), and NETS-activated microbeads were purchased from GE Healthcare (Little Chalfont, Buckinghamshire, United Kingdom). Dulbecco's phosphate-buffered saline (D-PBS), and streptavidin coupled magnetic beads (Dynabeads® M-270 Streptavidin) were purchased from Invitrogen (Carlsbad, Calif.).

The microfluidic device was fabricated using conventional multi-layer soft-lithography techniques. First, a layer of AZ-4620 positive photoresist (Clariant Corp. Somerville, N.J.) was spin-coated on a silicon wafer (Silicon Quest International, Inc., San Jose, Calif.), exposed to ultraviolet light through photomasks, developed, and baked to form the round-shaped flow channel that can be sealed completely. Then, using the same silicon wafer, SU-8 (MicroChem, Newton, Mass.) layers were spin-coated and developed to define the flow layer. In parallel, a layer of SU-8 photoresist was patterned on another silicon wafer to establish the control. Meanwhile, chrome (10 nm) and gold (100 nm) thin films were thermally evaporated on a glass slide, patterned through photolithograph, and wet etched to form heaters and temperature sensors. The heater and sensor were passivated by spin-coating PDMS prepolymer solution and curing at 72° C. for 30 minutes.

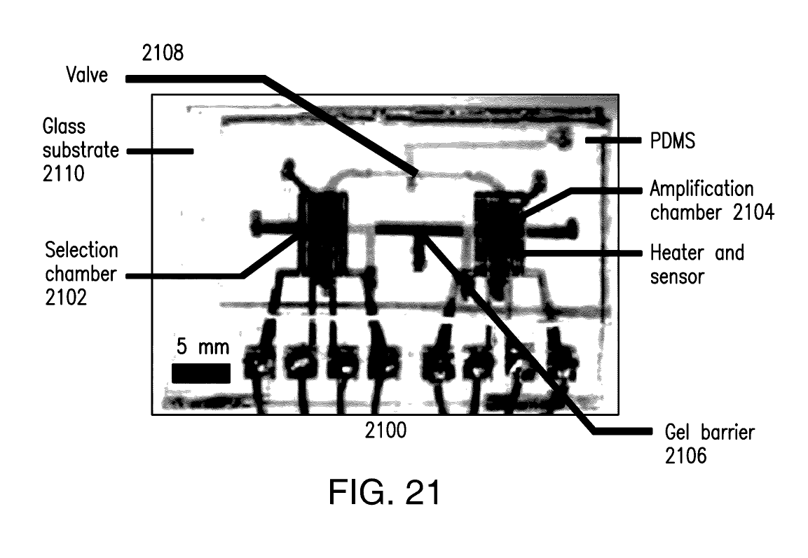

Subsequently, PDMS prepolymer solution (base and curing agent mixed in a 10:1 ratio) was spin-coated onto the silicon wafer bearing the flow layer, and cured on a hotplate at 72° C. for 15 minutes. Another PDMS prepolymer solution was cast onto the control layer silicon wafer and cured on a hotplate at 72° C. for 30 minutes. The resulting control layer PDMS slab was peeled off from the mold, punched to form a pneumatic inlet, and bonded to the PDMS membrane on the silicon mold bearing the flow layer features. The bonded slab was then peeled from the flow layer wafer. After punching inlets and outlets, the slab was bonded to a glass slide bearing the heater and temperature sensor. Finally, molten agarose gel was injected in the gel inlet of the device to and cured at room temperature to form the electrokinetic transfer channel. A fabricated device can be seen in FIG. 21. The fabricated device 2100 includes a selection chamber 2102 and an amplification chamber 2104. The selection chamber is connected to the amplification chamber via a first microchannel 2106 including a gel barrier and a second microchannel 2108 that includes a valve actuated by a pneumatic control channel. The microdevice was fabricated on a glass substrate 2110 and the selection chambers were created using PDMS.

NHS-activated microbeads are functionalized with protein by incubation with IgE. The functionalized microbeads are then introduced into the selection chamber of the device until approximately 40% of the selection chamber volume was occupied by beads. Selection of oligomers is then performed by infusing randomized library (1 uM) into the device (10 uL/min) for 10 minutes, followed by multiple washes with PBS buffer (20 uL/min) to remove weakly binding oligomers for 15 minutes. Next, primer functionalized magnetic beads are introduced into the amplification chamber of the device and held by an external magnet. Tris-boric acid electrolyte buffer is then injected into the device and platinum wires are inserted into the electrode inlets of each chamber with a 50 V potential difference applied between them for 35 minutes. Meanwhile, strongly bound oligomers remaining in the selection chamber are thermally eluted (50° C.) using the integrated heater and temperature sensor. The 25 V/cm electric field induced by the platinum wires electrokinetically transfers the thermally eluted oligomers to the positive electrode in the amplification chamber where the oligomers then hybridize to the reverse primers immobilized on the magnetic bead surfaces. The platinum wires are removed from the device eliminating the electric field, PCR reagents are introduced into the amplification chamber and bead-based PCR progresses utilizing the heater and temperature sensor located beneath the amplification chamber. A PCR process of 95° C. for 10 seconds, 59° C. for 30 seconds, and 72° C. for 10 seconds is used. After 20 cycles of PCR thermocycling, the IgE-functionalized microbeads are removed from the selection chamber and replaced with new IgE-functionalized microbeads. The valve is then opened and oligomers are released from the bead surfaces by heating to 95° C. The released oligomers are transported back to the selection chamber through the opened valve via pressure-driven flow (20 uL/min) for further affinity selection with the replenished microbeads. This closed-loop process is repeated for a total of four affinity selections and four 20-cycle PCR amplifications.

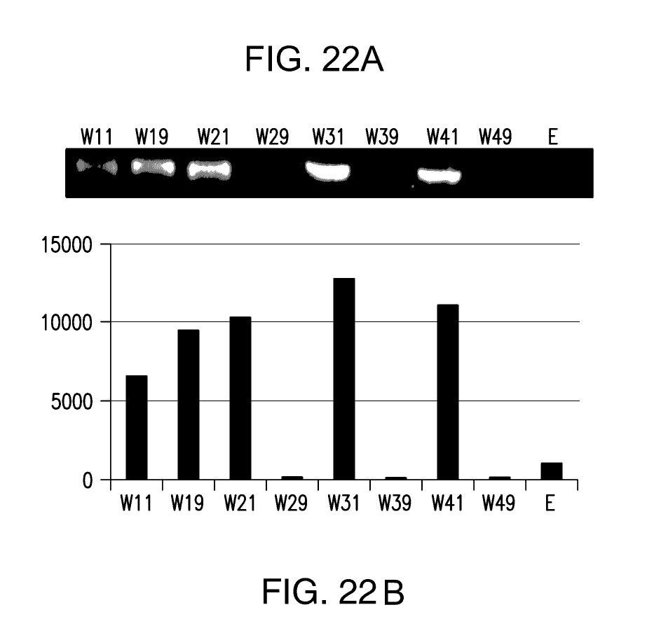

To demonstrate multi-round, closed-loop affinity selection and amplification, washing waste from four rounds of selection and the strongly bound thermally eluted ssDNA from the fourth round were collected, amplified (16 cycles PCR) and imaged with gel electrophoresis (FIGS. 22A-22B). Since the brightness of bands in a gel image represents the amount of oligomers in the eluent loaded in the lane, comparison of the band intensities allowed investigation of the selection process. In the first round some oligomers were in the washing waste after the completion of the washing process as indicated by the presence of a band in lane W19. However, the increase in band intensity from W19 (selection 1, wash 9) from W21 (selection 2, wash 1) suggests that oligomers were successfully eluted from the bead surfaces, electrokinetically transferred and amplified by PCR. The lack of a band in W29 (selection 2, wash 9) shows that weakly bound oligomers were removed from the washing process. The increase in band intensity in from W29 to W31 (selection 3, wash 1) and W39 (section 3, wash 9) to W41 (selection 4, wash 1) suggests that, again, oligomers were successfully thermally eluted, electrokinetically transferred to the amplification chamber, amplified by PCR, and transferred back to the selection chamber via pressure driven flow. The presence of a band in the elution lane (E), while W49 (selection 4, wash 9) lacks a band, suggests oligomers were strongly bound after four rounds of selection and amplification and were thermally eluted from the bead surfaces.

The enriched aptamer pool collected from the thermal elution of the fourth selection round was further investigated for its affinity and specificity using a fluorescence binding assay. Six different concentrations (100 nM, 50 nM, 25 nM, 12.5 nM, 6.25 nM and 3.125 nM) of fluorescently tagged oligomers (enriched aptamer pool or randomized library) were incubated with IgE-functionalized beads in triplicate 100 μL volumes. After incubating the oligomers with the beads for 30 minutes, the beads were washed and the bound oligomers were thermally eluted (95° C.). The eluted oligomers were collected and their relative amounts were determined with a Wallac EnVision Multilabel Reader fluorescent spectrometer.

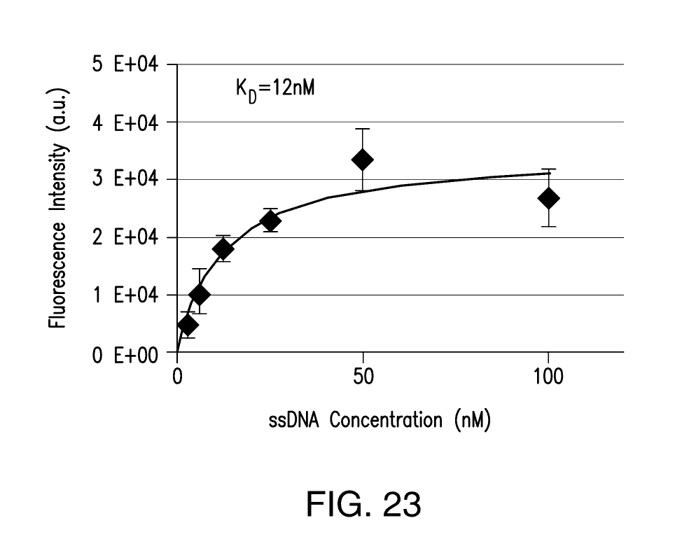

When the enriched pool was incubated with IgE-immobilized beads, washed, and bound oligomers were thermally eluted and measured, the fluorescent intensity rapidly increased until reaching an asymptote (FIG. 23). This indicated that the affinity of the enriched oligomer pool considerably improved after the microfluidic SELEX process. Assuming monovalent binding, the dissociation constant (KD) of the enriched pool was determined to be approximately 12 nM, which is consistent with that of existing IgE aptamers.

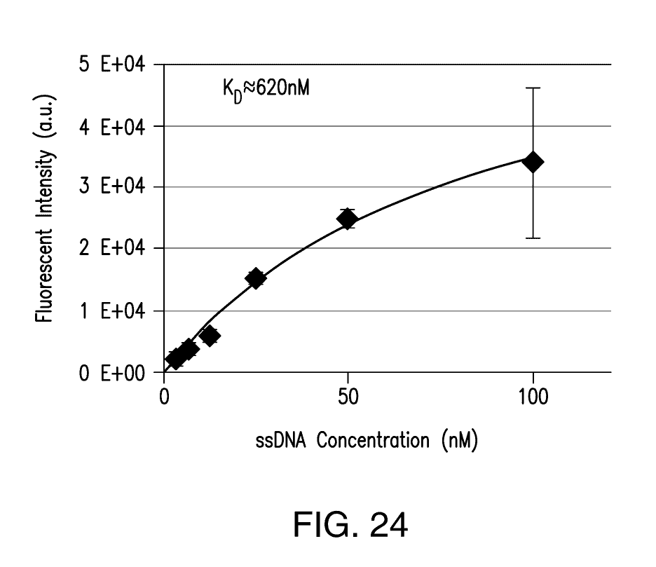

When the fluorescently tagged randomized library used to initiate aptamer selection was incubated with IgE-immobilized beads representing the amount of targetbound oligomers, increased without reaching an asymptote (FIG. 24). A nominal disassociation constant, also based on the assumption of monovalent binding, was estimated to be on the order of 620 nM. This is over 50 times the value of KD estimated for the enriched pool above, which suggests that there was negligible binding between the randomized library and IgE and demonstrates the ability of the microfluidic device to isolate aptamers against protein targets.

The description herein merely illustrates the principles of the disclosed subject matter. Various modifications and alterations to the described embodiments will be apparent to those skilled in the art in view of the teachings herein. Accordingly, the disclosure herein is intended to be illustrative, but not limiting, of the scope of the disclosed subject matter.

1. A method for selecting and isolating cell-targeting aptamers using a selection chamber comprising:
(a) culturing cells on a bottom portion of a selection chamber;
(b) introducing a first sample including a cell-targeting oligomer into the selection chamber, whereby the cell-targeting oligomer binds to the cultured cells; and
(c) removing unbound oligomers from the selection chamber to isolate the cell-targeting oligomer.

2. The method of claim 1, wherein the cells comprise cancer cells.

3. The method of claim 2, wherein the cancer cells comprise MCF-7 cells.

4. The method of claim 1, wherein the cell-targeting oligomer comprises single-stranded DNA.

5. The method of claim 1, wherein removing unbound oligomers from the selection chamber comprises infusing a washing buffer into the selection chamber.

6. The method of claim 1, further comprising:
(d) eluting the cell-targeting oligomer; and
(e) hydrodynamically transferring the cell-targeting oligomer to an amplification chamber.

7. The method of claim 6, wherein eluting the cell-targeting oligomer comprises adjusting the temperature in the selection chamber to weaken the bond between the cells and the cell-targeting oligomer.

8. The method of claim 7, wherein adjusting the temperature comprises controlling a heater.

9. The method of claim 6, wherein hydrodynamically transferring the cell-targeting oligomer comprises actuating one or more microvalves.

10. The method of claim 6, wherein the cell-targeting oligomer is transferred from the selection chamber to the amplification chamber via a first microchannel.

11. The method of claim 6, further comprising:
(e) providing primer-functionalized magnetic beads in the amplification chamber, the primer-functionalized magnetic beads configured to capture the cell-targeting oligomer; and
(f) amplifying the cell-targeting oligomer.

12. The method of claim 11, wherein the primer-functionalized magnetic beads are held in the amplification chamber using an external magnet.

13. The method of claim 11, wherein the primer-functionalized magnetic beads comprise streptavidin magnetic beads.

14. The method of claim 11, wherein amplifying the cell-targeting oligomer comprises applying a polymerase chain reaction technique.

15. The method of claim 11, further comprising hydrodynamically transferring the cell-targeting oligomer from the amplification chamber to the selection chamber.

16. The method of claim 6, wherein the selection chamber and the amplification chamber are formed on the a microchip.

17. A microdevice for selecting and isolating cell-targeting oligomers comprising a selection microchamber formed in a cavity of a multilayered thin film structure, the selection microchamber comprising cells immobilized on a bottom portion thereof, the microdevice made by culturing cells in the selection chamber.

18. The microdevice of claim 17, wherein the cells comprise cancer cells.

19. The microdevice of claim 18, wherein the cancer cells comprise MCF-7 cells.

20. The microdevice of claim 17, further comprising a heater configured to control the temperature in the selection chamber.

21. The microdevice of claim 20, wherein the heater comprises a resistive heater.

22. The microdevice of claim 20 wherein the heater is serpentine-shaped.

23. The microdevice of claim 20, further comprising a temperature sensor.

24. The microdevice of claim 17, further comprising an amplification chamber coupled to the selection chamber via a microchannel.

25. The microdevice of claim 24, further comprising one or more microvalves configured to hydrodynamically transfer cell-targeting oligomers from the selection chamber to the amplification chamber.

26. The microdevice of claim 25, further comprising a pneumatic control channel configured to actuate the one or more microvalves.

27. The microdevice of claim 26, wherein the pneumatic control channel comprises an oil-filled channel.

28. The microdevice of claim 24, wherein the amplification chamber comprises a plurality of primer-functionalized magnetic beads.

29. The microdevice of claim 28, wherein the primer-functionalized magnetic beads comprise streptavidin-coated magnetic beads.

30. The microdevice of claim 28, further comprising an external magnet positioned to hold the primer-functionalized microbeads in the amplification chamber.

31. The microdevice of claim 24, further comprising a first resistive heater located under the selection channel and a second resistive heater located under the amplification channel.

32. The microdevice of claim 24, further comprising a first microchannel comprising one or more microvalves configured to hydrodynamically transfer cell-targeting oligomers from the selection chamber to the amplification chamber and a second microchannel comprising one or more microvalves configured to hydrodynamically transfer cell-targeting oligomers from the amplification chamber to the selection chamber.

33. A method for isolating and amplifying an aptamer using a selection chamber and an amplification chamber, comprising:
(a) introducing a first sample comprising an oligomer into the selection chamber;
(b) isolating the oligomer;
(c) hydrodynamically transferring the oligomer from the selection chamber to the amplification chamber; and
(d) amplifying the oligomer.

34. The method of claim 33, further comprising hydrodynamically transferring the oligomer from the amplification chamber to the selection chamber.

35. A method for isolating and amplifying an aptamer using a selection chamber and an amplification chamber, comprising:
(a) introducing a first sample comprising an oligomer into the selection chamber;
(b) isolating the oligomer;
(c) transferring the oligomer from the selection chamber to the amplification chamber;
(d) amplifying the oligomer in the amplification chamber; and
(e) hydrodynamically transferring the oligomer from the amplification chamber to the selection chamber.

36. The method of claim 35, wherein the selection chamber comprises cultured cells.

37. The method of claim 35, wherein the selection chamber comprises microbeads.

38. The method of claim 37, wherein the microbeads comprise Immunoglobin-E functionalized microbeads.

39. The method of claim 37, wherein the microbeads are retained in the selection chamber by a weir structure.

40. The method of claim 35, wherein the oligomer is transferred from the selection chamber to the amplification chamber via a first microchannel.

41. The method of claim 40, wherein the oligomer is hydrodynamically transferred from the amplification chamber to the selection chamber via a second microchannel.

42. The method of claim 35, wherein the oligomer is transferred from the amplification chamber to the selection chamber via electrophorsesis.

43. The method of claim 35, wherein the oligomer is hydrodynamically transferred from the amplification chamber to the selection chamber.

44. The method of claim 35, wherein the amplification chamber comprises primer-functionalized magnetic beads.

45. The method of claim 35, wherein hydrodynamically transferring the oligomer comprises actuating one or more microvalves.

46. A microdevice for selecting and isolating cell-targeting aptamers comprising:
a selection microchamber;
an amplification microchamber;
a first microchannel between the selection chamber and the amplification chamber configured to transfer oligomers from the selection chamber to the amplification chamber; and
a second microchannel between the selection chamber and the amplification chamber configured to transfer oligomers from the amplification chamber to the selection chamber,
wherein at least one of the first microchannel and the second microchannel comprises one or more microvalves.

47. The microdevice of claim 46, wherein the selection chamber comprises cultured cells.

48. The microdevice of claim 46, wherein the selection chamber comprises microbeads retained on a weir structure.

49. The microdevice of claim 46, further comprising a heater and a temperature sensor positioned below the selection chamber.

50. The microdevice of claim 46, wherein the first microchannel comprises the one or more microvalves configured to hydrodynamically transfer the oligomer from the selection chamber to the amplification chamber.

51. The microdevice of claim 50, further comprising a pneumatic control channel configured to actuate the one or more microvalves.

52. The microdevice of claim 51, wherein the pneumatic control channel comprises an oil-filled channel.

53. The microdevice of claim 46, wherein the amplification chamber comprises primer-functionalized microbeads.

54. The microdevice of claim 53, further comprising an external magnet positioned to hold the primer-functionalized microbeads beads in the amplification chamber.

55. The microdevice of claim 46, wherein the second microchannel comprises the one or more microvalves configured to hydrodynamically transfer the oligomer from the amplification chamber to the selection chamber.

56. The microdevice of claim 55, further comprising a pneumatic control channel configured to actuate the one or more microvalves.

57. The microdevice of claim 56, wherein the pneumatic control channel comprises an oil-filled channel.

58. The microdevice of claim 55, wherein the first microchannel comprises an agarose gel.
